(19) United States
(12) Patent Application Publication
(10) Pub. No.: US 2022/0267857 A1
(43) Pub. Date: Aug. 25, 2022

(54) METHOD FOR DETERMINING THE RESPONSE TO TREATMENT OF A PATIENT AFFECTED BY NON-SMALL CELL LUNG CARCINOMA (NSCLC)

(71) Applicant: FUNDACIÓN PARA LA INVESTIGACIÓN BIOMÉDIKCA DEL HOSPITAL UNIVERSITARIO LA PAZ (FIBHULP), Madrid (ES)

(72) Inventors: Inmaculada IBÁÑEZ DE CÁCERES, Madrid (ES); Javier DE CASTRO CARPEÑOñ, Madrid (ES); Julia JIMÉNEZ HERNÁNDEZ, Madrid (ES); Carlos RODRÍGUEZ ANTOLÍN, Madrid (ES); Olga VERA PUENTE, Madrid (ES); Rocío ROSAS ALONSO, Madrid (ES); Olga PERNÍA ARIAS, Madrid (ES); Itsaso LOSANTOS GARCÍA, Madrid (ES)

(21) Appl. No.: 17/628,179

(22) PCT Filed: Jul. 10, 2020
(86) PCT No.: PCT/EP2020/069659

§ 371 Date: Jan. 18, 2022

(30) Foreign Application Priority Data
Jul. 19, 2019 (EP) 19382614.6

(51) Int. Cl. C12Q 1/6886

(52) U.S. Cl. CPC C12Q 1/6886; C12Q 2600/112; C12Q 2600/118; C12Q 2600/158; C12Q 2600/178

(57) ABSTRACT

This invention refers to the medical field, in particular, the prediction of progression or response to treatment of a patient affected by Non-small cell lung carcinoma (NSCLC); more particularly the in vitro use of miRNA molecules isolated from the inside of circulating exosomes of a sample isolated from serum, blood or plasma obtained or isolated from a human subject.

### TECHNICAL FIELD OF THE INVENTION

This invention refers to the medical field, in particular, the prediction of progression or response to treatment of a patient affected by Non-small cell lung carcinoma (NSCLC); more particularly the in vitro use of miRNA molecules isolated from the inside of circulating exosomes of a sample isolated from serum, blood or plasma obtained or isolated from a human subject.

### BACKGROUND OF THE INVENTION

Non-small cell lung carcinoma (NSCLC) is one of the most frequent type of cancer worldwide (11.6% of new cases in 2018) and, by far, the most lethal (18.2% of all cancer-related deaths. The advanced stage of the disease at the time of diagnosis and the innate or acquired anti-cancer drug resistance are the main causes of this high mortality.

Several mutations have been identified in lung cancer, such as in EGFR, ALK, BRAF or ROS1, that represent robust predictive biomarkers as well as very valuable therapeutic targets. However, only between 15 to 30% of the cases have these targetable driven mutations. Thus, in advanced stages with disseminated disease, which are not eligible for surgical resection, the standard treatment remains platinum-based chemotherapy. Unfortunately, as we mentioned above, in many cases there is a lack of response to the treatment, which leads to an early relapse and a decrease in the overall survival of these patients. For this reason, it is very important to identify new biomarkers for survival, prognosis, and drug-resistance in NSCLC, to be able to molecularly adapt the treatments to each patient.

Exosomes are small-sized vesicles (30-150 nm) with an endosomal origin that are released by fusion of the multivesicular bodies with the plasmatic membrane. These nanovesicles are released by most cell types as a mechanism of cellular communication, so they are found in different biological fluids such as blood, urine, saliva or cerebrospinal fluid. Exosomes are considered as “information shuttles” because they contain biologically active molecules such as proteins, RNA and DNA, which are capable of generating changes in the target cell after the internalization. In fact, in the last decades numerous studies have shown the involvement of exosomes in diverse contexts, both physiological and pathogenic. However, its role in cancer has gained special relevance in recent years, since tumor cells release a large amount of exosomes compared with normal cells. Several studies describe their participation in tumorigenic processes such as immune system evasion, angiogenesis, premetastatic niche development or resistance to anti-cancer drugs. Many researchers have focused their studies on exosomal miRNAs and proteins, since they are molecules that generate changes in a post-translational level. In 2007, Valadi described, for the first time, that miRNAs are present in exosomes.

### BRIEF DESCRIPTION OF THE FIGURES

FIG. 1. Viability curves to CDDP and CBDCA. The curves show CDDP resistance of cells (A) H23R, (B) H460R, (C) A2780R and (D) 41MR; and to CBDCA of cells (E) H23R-CBDCA and (F) A2780R-CBDCA. The data was normalized with respect to the untreated control, which was established as 100%. IC50 is the concentration that induces the death of 50% of the cell population. The resistance index (IR) is calculated as IC50 of the resistant cell line/IC50 of the sensitive cell line. P<0.001 was considered a significant change in drug sensitivity (Student's t test).

FIG. 2. Characterization of the exosomes secreted by the cisplatin-resistant subtypes of the lung cancer lines H23R and H460R; and of ovarian cancer A2780R and 41MR. (A) Visualization of the exosomes with transmission electron microscopy. Images taken at 120,000 magnifications. (B) Determination of the size and concentration of exosomes by Nanosight (NTA).

FIG. 3. Viability and internalization analysis after incubation of exosomes from the secretome of CDDP-resistant lines with sensitive cell lines by flow cytometry. The exosomes of the resistant cells were labeled with PKH26 and the sensitive cells with CTV. (A) H23, (B) H460, (C) A2780 and (D) 41M. The quadrants delimit the positive or negative character of the marking signal on each axis. In all cases, PBS labeled with PKH26 was used as control. Viability was evaluated with compound 7AAD, which binds to dead cells. The internalization data were calculated on the percentage of living cells.

FIG. 4. Viability assays of response to cisplatin. Viability curves to CDDP in sensitive cells incubated with exosomes from each resistant subtype in (A) 41M cells after 24, 48 and 72 h of incubation and (B, C and D) in H23, A2780 and H460 cells after 72 h of incubation respectively. The data of each cell line was normalized with respect to the untreated control, which was established as 100%. The viability curves of the resistant subtypes (orange) are also shown.

FIG. 5. Selection of over-represented miRNAs in exosomes from cells with CDDP-resistant phenotype in comparison with those from sensitive cells. The data were obtained by massive sequencing by small RNAseq and were selected by two routes depending on whether they were known miRNAs (A) or new ones (B).

FIG. 6. Relative levels of exosomal miRNAs quantified by qRT-PCR with TaqMan™ probes. Levels are shown on paired sensitive and CDDP-resistant lines of NSCLC H23 (A) and ovarian cancer A2780 (B) and 41M (C), as well as in the paired lines sensitive and resistant to CBDCA H23 (D) and A2780 (E). The levels of the sensitive phenotype were used as a calibrator in all cases and represent the mean±standard deviation of at least 3 independent experiments performed in triplicate for each cell line analyzed. The miR-151a was used as endogenous exosomal miRNA for normalization. The absence of bars indicates that there was no amplification in the PCR.

FIG. 7. Selection of exosomal proteins promoting CDDP resistance. Selection from the data obtained after the shotgun proteomic analysis in lung cancer cell lines H23S/R and ovarian cancer A2780S/R and 41M/MR.

FIG. 8. Survival analysis according to the levels of each candidate miRNA. Kaplan-Meier curves of progression-free survival (left) and overall survival (right) in the prospective cohort of 51 NSCLC patients separated by having high or low levels according to the 75th percentile of the miR-142-3p (A), miR-451a (B) and according to the 25th percentile of miR-55745 (C) in circulating plasma exosomes.

FIG. 9. Comparison of miR-novel-55745 levels between healthy controls (N=10) and patients with NSCLC (N=51). *=p<0.01 Student's t test.

FIG. 10. Survival analysis according to the levels of miR-142-3p and 451a. Kaplan-Meier curves of progression-free survival (left) and overall survival (right) in the prospective cohort of 51 NSCLC patients separated by jointly having high or low levels, according to the 75th percentile, of miR-142-3p and miR-451a in circulating plasma exosomes.

FIG. 11. Survival analysis according to the levels of each candidate protein. Kaplan-Meier curves of progression-free survival (left) and overall survival (right) in the cohort of 45 NSCLC patients separated by having high or low levels of the ITIH1 (A), FBN2 (B) and LOXL2/GSN (C) in circulating plasma exosomes.

### DESCRIPTION OF THE INVENTION

### Definitions

For the purpose of the present invention, the following definitions are included below:
The term “screening” is understood as the examination or testing of a group of individuals diagnosed with Non-small cell lung carcinoma and treated with a drug selected from the list consisting of: radiotherapy, immunotherapy, chemotherapy, a DNA alkylating agent, a DNA cross-linking agent, or any combination thereof, with the objective of discriminating between individuals with poor overall survival or poor progression free survival from those individuals with good overall survival or good progression free survival.
The term “Non-small cell lung carcinoma or NSCLC” is any type of epithelial lung cancer other than small cell lung carcinoma (SCLC). NSCLC accounts for about 85% of all lung cancers. As a class, NSCLCs are relatively insensitive to chemotherapy, compared to small cell carcinoma.
The term “advanced non-small cell lung carcinoma or advanced NSCLC” is understood as stage III or stage IV non-small cell lung carcinoma.
The term “Progression-free survival (PFS)” is the length of time during and after the treatment of a disease, such as cancer, that a patient lives with the disease but it does not get worse. Preferably, the term “poor PFS” is understood as median of progression free survival less than 5 months.
The term “overall survival (OS)” is stated as a five-year survival rate, which is the percentage of people in a study or treatment group who are alive five years after their diagnosis or the start of treatment. Also called survival rate. The term “poor OS” is understood as median of overall survival less than 5 months.
The term “relapse” is understood as time from the start of treatment until the tumor grows
The term “exitus” is understood as death.
The expression “minimally-invasive biological sample” refers to any sample which is taken from the body of the patient without the need of using harmful instruments, other than fine needles used for taking the blood from the patient, and consequently without being harmfully for the patient. Specifically, minimally-invasive biological sample refers in the present invention to: blood, serum, or plasma samples.
The term “up-regulated” of any of the micro-RNAs or combinations thereof described in the present invention, refers to an increase in their levels or concentration with respect to a given “threshold value” or “cutoff value”, by at least 5%, by at least 10%, by at least 15%, by at least 20%, by at least 25%, by at least 30%, by at least 35%, by at least 40%, by at least 45%, by at least 50%, by at least 55%, by at least 60%>, by at least 65%>, by at least 70%, by at least 75%, by at least 80%, by at least 85%, by at least 90%, by at least 95%, by at least 100%, by at least 110%, by at least 120%, by at least 130%, by at least 140%, by at least 150%, or more.
The term “threshold value” or “cutoff value”, when referring to the levels or concentrations of the miRNAs described in the present invention, refers to a reference level or concentration indicative that a subject is likely to have a poor overall survival or poor progression free survival if the levels of the patient are above said threshold or cut-off or reference levels.
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.
It is also noted that the term “kit” as used herein is not limited to any specific device and includes any device suitable for working the invention such as but not limited to microarrays, bioarrays, biochips or biochip arrays.

A variety of statistical and mathematical methods for establishing the threshold or cutoff level are known in the prior art. A threshold or cutoff level for a particular biomarker may be selected, for example, based on data from Receiver Operating Characteristic (ROC) plots, as described in the Examples and Figures of the present invention. One of skill in the art will appreciate that these threshold or cutoff levels can be varied, for example, by moving along the ROC plot for a particular biomarker or combinations thereof, to obtain different values for sensitivity or specificity thereby affecting overall assay performance. For example, if the objective is to have a robust prognostic method from a clinical point of view, we should try to have a high sensitivity. However, if the goal is to have a cost-effective method we should try to get a high specificity. The best cutoff refers to the value obtained from the ROC plot for a particular biomarker that produces the best sensitivity and specificity. Sensitivity and specificity values are calculated over the range of thresholds (cutoffs). Thus, the threshold or cutoff values can be selected such that the sensitivity and/or specificity are at least about 70%, and can be, for example, at least 75%, at least 80%, at least 85%, at least 90%, at least 95%, at least 96%, at least 97%, at least 98%, at least 99% or at least 100% in at least 60% of the patient population assayed, or in at least 65%, 70%, 75% or 80% of the patient population assayed.

Consequently, each of the embodiments cited through-out the present invention is preferably carried out by determining the levels of at least the micro-RNAs cited in the present invention in a sample isolated from a subject to be screened, and comparing the levels of said micro-RNAs with predetermined threshold or cutoff values, wherein said predetermined threshold or cutoff values correspond to the level of said micro-RNAs which correlates with the highest specificity at a desired sensitivity in a ROC curve calculated based on the levels of the micro-RNAs determined in a patient population diagnosed with Non-small cell lung carcinoma, wherein the up regulation of at least one of said micro-RNAs with respect to said predetermined cutoff value is indicative that the subject shall have a poor OS or PFS.
miR-142-3p or miR-142 is understood as the 3′ arm of microRNA 142 [Homo sapiens (human)] with Gene ID: 406934.
miR-451a is understood as microRNA 451a [Homo sapiens (human)] with Gene ID: 574411
miR-55745 is understood as a microRNA [Homo sapiens (human)] with the following mature sequence: agugaaaugacuugagagg (SEQ ID NO 1) and chromosome location according with hg38 coordinates: Chr4:76846064-76846129.
ITIH1, LOXL2, GELSOLIN, FBN2 are respectively understood as proteins Inter-alpha-trypsin inhibitor heavy chain H1, Lisil oxidase homolog 2, Gelsolin and Fibrillin 2 with Uniprot ID: ID: P19827, Q9Y4KO, P06396 and P35556.

### DESCRIPTION OF THE INVENTION

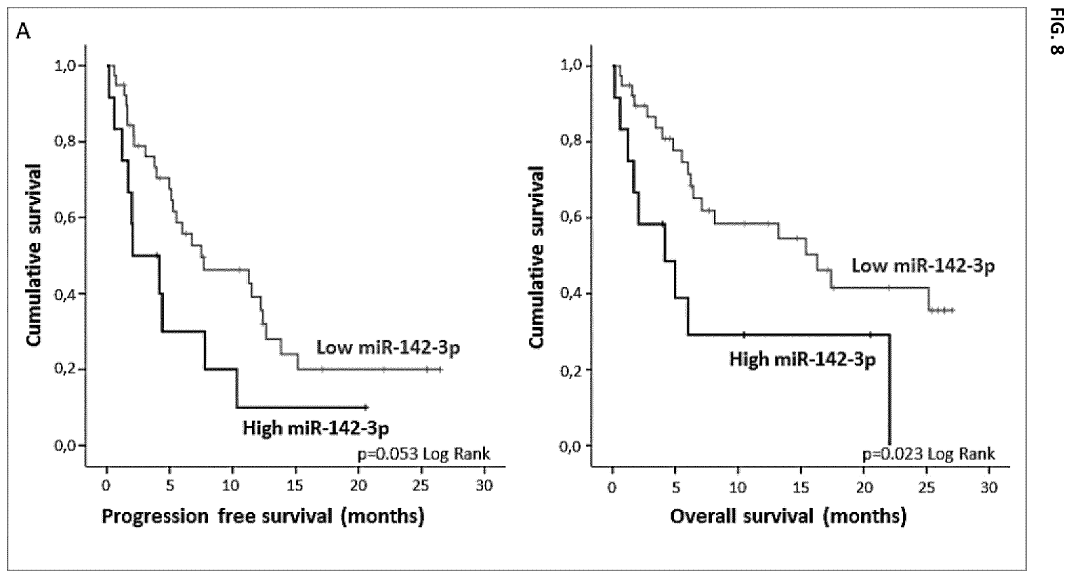

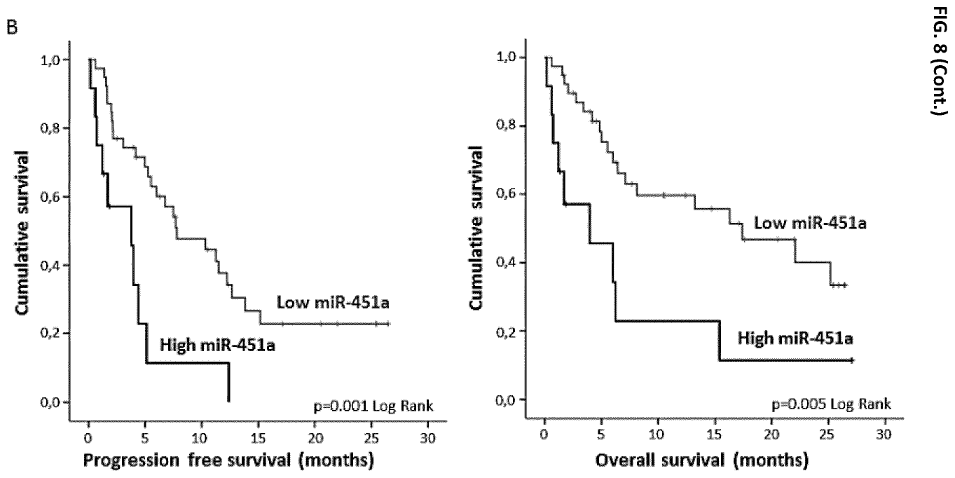

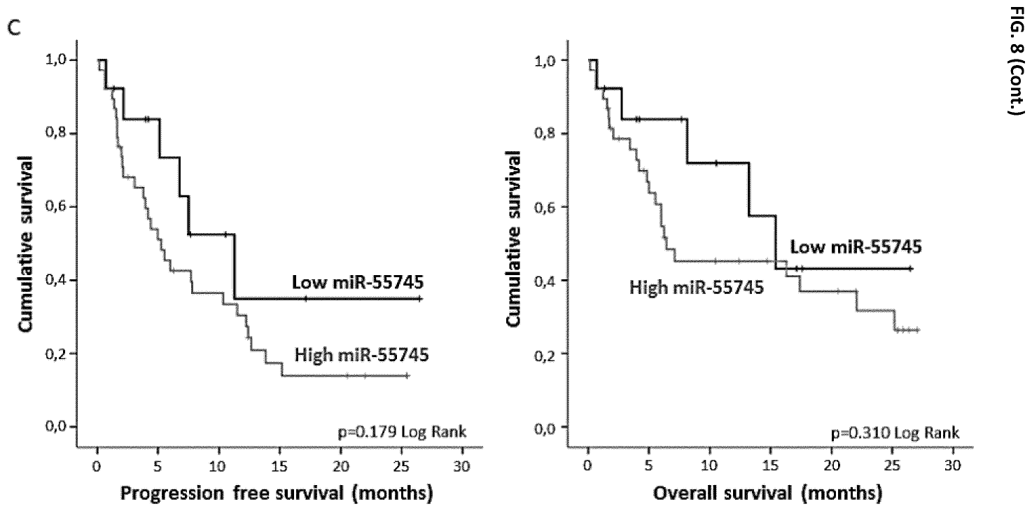

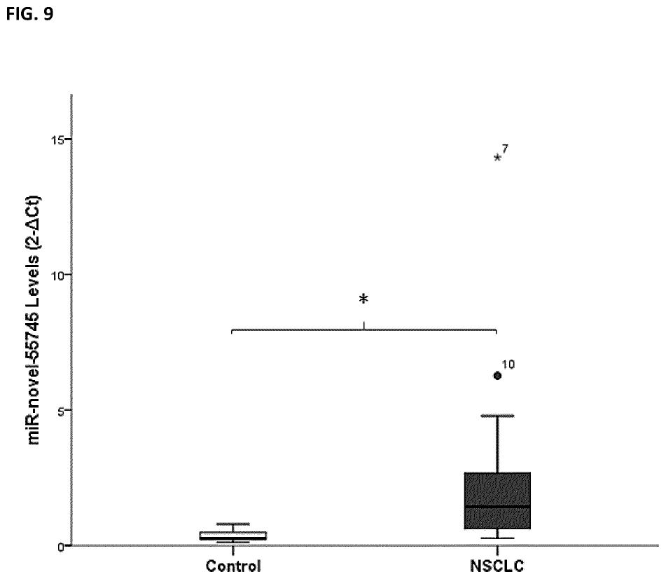

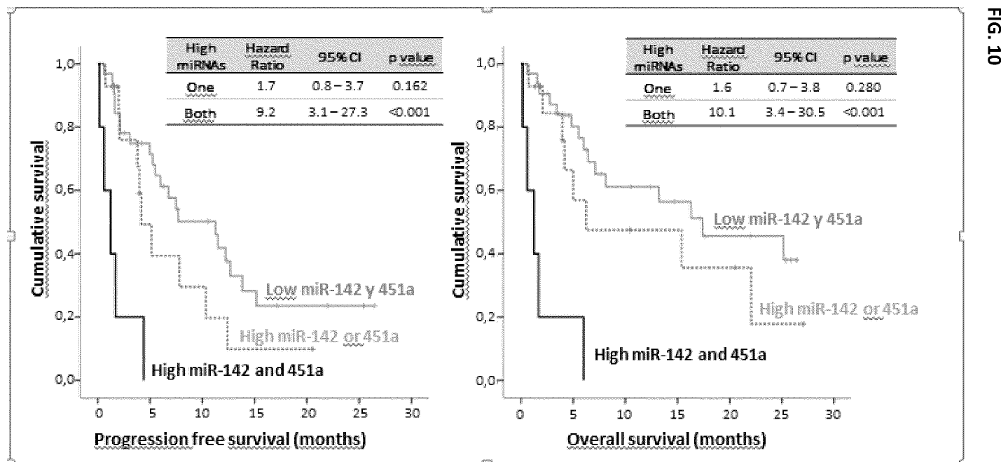

In the present study we show that high levels of, among others biomarkers, exosomal miR-142-3p and miR-451a increase the risk of recurrence and death in NSCLC patients. We also show that high levels of exosomal proteins ITIH1, LOXL2, Gelsolin and FBN2 can predict the prognosis in NSCLC. In particular, and as indicated in the examples of the present specification, in order to determine the clinical relevance of the present findings, we collected 51 plasma samples from advanced NSCLC patients (stages III and IV) who had not received any treatment at the time of extraction. After that, all of them received platinum-based chemotherapy as first line of treatment and were clinically followed in order to monitor the time of relapse and overall survival. On the one hand, we extracted total exosomal RNA and analyzed the levels of the miRNAs validated in H23 lung cancer cell line: miR-142, miR-451a and miR-55745 in our NSCLC cohort by qRT-PCR. We divided patients into two groups: high or low levels of the miRNA each case. The cut-off was optimized by performing a COX regression analysis segmenting the cohort in the different quartiles, finally selecting the patients “high-miRNA” and “low-miRNA” according to their position with respect to the 75 percentile in each of the miRNAs. To evaluate the association between miRNAs' levels and prognosis in NSCLC patients we used Kaplan-meier survival analysis. We found that high levels of exosomal miR-142 were significantly related with a poor overall survival and progression-free survival (PFS) (FIG. 8A). We also found that the patients with higher levels of exosomal miR-451a showed poorer prognosis in terms of PFS and OS (FIG. 8B). Regarding the unknown miR-55745, we found a statistically significant increase (p=0.024) in exosomal miRNA-55745 levels in advanced NSCLC patients compared with levels obtained in a cohort of 10 healthy controls (FIG. 9). Furthermore, to investigate the strength of the combination of both miRNAs 142 and 451a in the prognosis prediction, we found that the risk of relapse increases more than 9 times and the risk of exitus more than 10 times in patients with high levels of both miRNAs (FIG. 10).

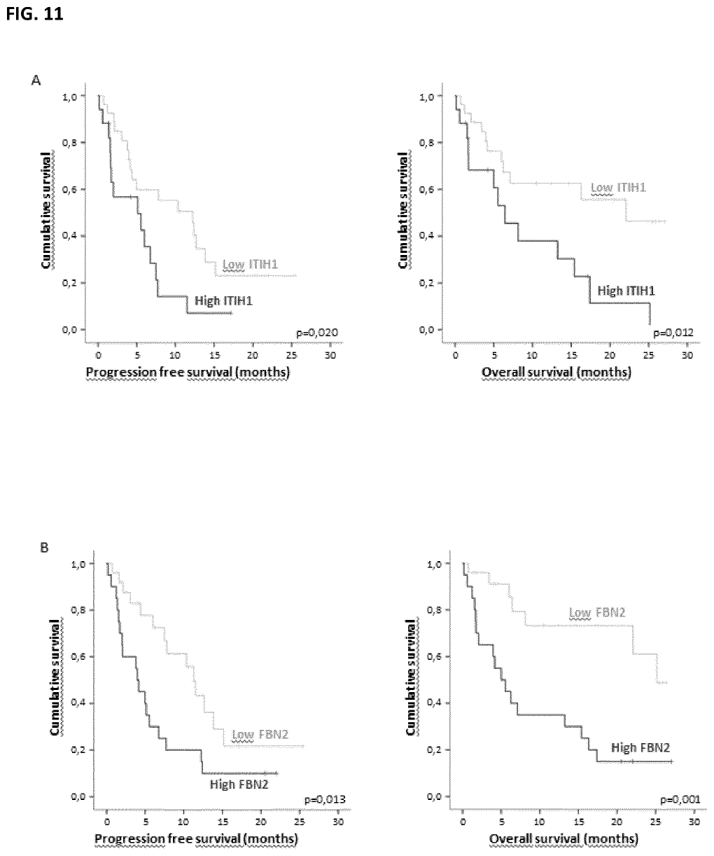

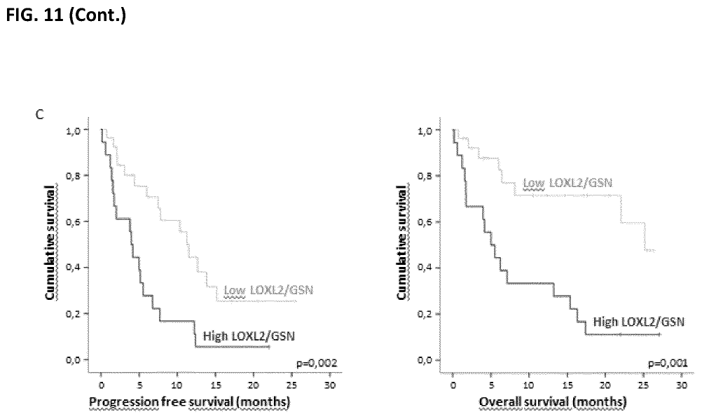

On the other hand, we extracted exosomal proteins from 45 samples of our cohort of advanced NSCLC patients by size exclusion chromatography. We performed a Targeted Mass Spectrometry Analysis of the 20 selected proteins in the cell lines and we found presence of 10 of them inside the circulating exosomes from the patients. In the same way as with the miRNAs, we divided patients into 2 groups: with high or with low levels of each protein, and performed Kaplan-meier survival analysis to evaluate their implication in prognosis. The cut-off was determined as the median of the protein level each case. Our results showed that high levels of the protein ITIH1 were significantly related with a poor OS and PFS. We also found that proteins LOXL2, GELSOLIN and FBN2 presented the same distribution between patients, and that high levels of them were correlated with a poorer prognosis in terms of PFS and OS (FIG. 11A-C).

Therefore, a first aspect of the invention refers to the in vitro use of the levels of miR-142, miR-451a, or any combination thereof, isolated or present inside circulating exosomes of a sample isolated from serum, blood or plasma of a human subject diagnosed with Non-small cell lung carcinoma, for the purpose of predicting progression-free survival in that subject or to predict the overall survival of the subject. More preferably, the subject suffering from Non-small cell lung carcinoma has not been treated yet or is preferably being treated with a drug selected from the list consisting of: radiotherapy, immunotherapy, chemotherapy, a DNA alkylating agent, a DNA cross-linking agent, or any combination thereof.

It is noted that the upregulation of the levels of miR-142, miR-451a, or any combination thereof, isolated or present inside the circulating exosomes is indicative of a poor overall survival or of a poor PFS. Moreover, it is further noted that the upregulation of the levels of miR-142 and miR-451a isolated or present inside the circulating exosomes is indicative of an increased risk of relapse and an increased risk of exitus.

Therefore, a preferred embodiment of the first aspect of the invention, refers to the in vitro use of the levels of both miR-142 and miR-451a, isolated or present inside circulating exosomes of a sample isolated from serum, blood or plasma of a human subject diagnosed with Non-small cell lung carcinoma, for the purpose of predicting the risk of relapse and or exitus in said subject.

It is noted that the prediction of the first aspect of the invention, or of any preferred embodiments therefrom, is determined by comparing the levels of miR-142, miR-451a, or any combination thereof with a threshold or cutoff level, wherein preferably such level is obtained from a group of healthy controls or corresponds to the, preferably 75th percentile value, of the normalized levels of the amount of exosomal miR-142-3p and miR-451a (21.35 2^{-ΔCt} and 1258.07 2^{-ΔCt} respectively) versus miR-151a in NSCLC patients.

A second aspect of the invention refers to the in vitro use of the levels of miRNA-55745, isolated or present inside circulating exosomes of a sample isolated from serum, blood or plasma of a human subject diagnosed with Non-small cell lung carcinoma, for the purpose of predicting the risk that the subject suffers from advanced NSCLC, when the levels of miRNA-55745 are compared with levels obtained in a group of healthy controls. Alternatively, the second aspect of the invention refers to the in vitro use of the levels of miRNA-55745, isolated or present inside circulating exosomes of a sample isolated from serum, blood or plasma of a human subject, for the purpose of indicating the risk that the subject suffers from NSCLC, preferably from early NSCLC, when the levels of miRNA-55745 are compared with levels obtained in healthy controls. More preferably, the subject suffering from Non-small cell lung carcinoma has not been treated yet or is preferably being treated with a drug selected from the list consisting of: radiotherapy, immunotherapy, chemotherapy, a DNA alkylating agent, a DNA cross-linking agent, or any combination thereof.

It is noted that the prediction of the second aspect of the invention is determined by comparing the levels of miRNA-55745, with a threshold or cutoff level, wherein preferably such level is obtained from a group of healthy controls (as already indicated above), or corresponds to, preferably the 25th percentile value of, the normalized levels of the amount of exosomal miR-55745 (2.69 2^{-ΔCt}) versus miR-151a in NSCLC patients.

A third aspect of the invention refers to the in vitro use of the levels of ITIH1, LOXL2, GELSOLIN, FBN2, or any combination thereof, isolated or present inside circulating exosomes of a sample isolated from serum, blood or plasma of a human subject diagnosed with Non-small cell lung carcinoma, for the purpose of predicting progression-free survival in that subject or to predict the overall survival of the subject. More preferably, the subject suffering from Non-small cell lung carcinoma has not been treated yet or is preferably being treated with a drug selected from the list consisting of: radiotherapy, immunotherapy, chemotherapy, a DNA alkylating agent, a DNA cross-linking agent, or any combination thereof.

It is noted that the upregulation of the levels of ITIH1, LOXL2, GELSOLIN, FBN2, or any combination thereof, isolated or present inside the circulating exosomes is indicative of a poor overall survival or of a poor PFS. It is further noted that the prediction of the third aspect of the invention is determined by comparing the levels of ITIH1, LOXL2, GELSOLIN, FBN2, or any combination thereof, with a threshold or cutoff level, wherein preferably such level is obtained from a group of healthy controls or corresponds to the mean value of intensity of each protein (88006.8; 5733630.8; 352569 and 355354.2 Arbitrary Unit of Intensity, respectively) in NSCLC patients.

More preferably, the use of any of the first or third aspects of the invention, or of any preferred embodiments therefrom, is done for the purpose of monitoring the disease of a subject who has NSCLC and who is preferably being treated with a drug selected from the list consisting of: radiotherapy, immunotherapy, chemotherapy, a DNA alkylating agent, a DNA cross-linking agent, or any combination thereof. Such determination is performed on the basis of the prediction of the progression-free survival or overall survival of the subject.

More preferably, the use of the first or third aspects of the invention, or of any preferred embodiments therefrom, is done in order to determine the response to treatment of a subject suffering from NSCLC and who is preferably being treated with a drug selected from the list consisting of: radiotherapy, immunotherapy, chemotherapy, a DNA alkylating agent, a DNA cross-linking agent or any combination thereof. Even more preferably, this alkylating agent, according to any of the above preferred embodiments, is selected from the list consisting of nitrogen mustards such as mechlorethamine, cyclophosphamide, ifosfamide, melphalan, or chlorambucil; ethyleneimines and methylmelamines such as altretamine; methylhydrazine derivatives such as procarbazine; alkyl sulfonates such as busulfan; nitrosoureas such as carmustine; triazenes such as dacarbazine or temozolomide; or platinum coordination complexes such as cisplatin, carboplatin, oxaliplatin, dicycloplatin, eptaplatin, lobaplatin, Miriplatin, Nedaplatin, Oxaliplatin, Picoplatin, Satraplatin, Triplatin or tetranitrate. Such determination of the response to treatment is performed on the basis of the prediction of the progression-free survival or overall survival of the subject.

In a particular embodiment of any of the first to third aspects of the invention, or of any preferred embodiments therefrom, such circulating exosomes from a sample isolated from serum, blood, or plasma are of tumor origin. In this sense, it should be noted that there are specific kits that allow DNA from circulating exosomes to be obtained from a sample isolated from serum, blood or plasma of tumour origin (Exosomics).

A fourth aspect of the invention refers to a method to monitor the progression of the oncological disease of a subject diagnosed with NSCLC, wherein such a method comprises the following steps:
a. Performing the methodology to determine the levels of miR-142, miR-451a, or any combination thereof, isolated or present inside circulating exosomes of a sample isolated from serum, blood or plasma of a human subject diagnosed with Non-small cell lung carcinoma; and
b. Determining the subject's disease monitoring or progression based on determination carried out in step (a),

wherein the upregulation of the levels of miR-142 or miR-451a isolated or present inside the circulating exosomes is indicative of a poor overall survival or of a poor PFS of the subject, and wherein the upregulation of the levels of miR-142 and miR-451a isolated or present inside the circulating exosomes is indicative of an increased risk of relapse and an increased risk of exitus.

It is further noted that for monitoring the progression of the disease of the fourth aspect of the invention, or of any preferred embodiments therefrom, such determination is made by comparing the levels of miR-142, miR-451a, or any combination thereof, with a threshold or cutoff level, wherein preferably such level is obtained from a group of healthy controls or corresponds to, preferably the 75th percentile value of, the normalized levels of the amount of exosomal miR-142-3p and miR-451a (21.35 2^{-ΔCt} and 1258.07 2^{-ΔCt} respectively) versus miR-151a in NSCLC patients.

A fifth aspect of the invention refers to a method to monitor the progression of the oncological disease of a subject diagnosed with NSCLC or to aid at the diagnosis of NSCLC, wherein such a method comprises the following steps:
a. Performing the methodology to determine the levels of miRNA-55745, isolated or present inside circulating exosomes of a sample isolated from serum, blood or plasma of a human subject diagnosed with Non-small cell lung carcinoma or suspected of suffering from NSCLC; and
b. Determining the subject's disease, diagnosis, monitoring or progression based on determination carried out in step (a),

wherein the upregulation of the levels of miRNA-55745 isolated or present inside the circulating exosomes is indicative that the subject suffers from NSCLC, in particular the subject may suffer from advanced NSCLC, when the levels of miRNA-55745 are compared with levels obtained in healthy controls.

It is further noted that for predicting the risk that the subject suffers from advanced NSCLC of the fifth aspect of the invention, or of any preferred embodiments therefrom, such determination is made by comparing the levels of miRNA-55745, with a threshold or cutoff level, wherein preferably such level is obtained from a group of healthy controls or corresponds to, preferably the 25th percentile value of, the normalized levels of the amount of exosomal miR-55745 (2.69 2^{-ΔCt}) versus miR-151a in NSCLC patients.

A sixth aspect of the invention refers to a method to monitor the progression of the oncological disease of a subject diagnosed with NSCLC, wherein such a method comprises the following steps:
a. Performing the methodology to determine the levels of ITIH1, LOXL2, GELSOLIN, FBN2, or any combination thereof, isolated or present inside circulating exosomes of a sample isolated from serum, blood or plasma of a human subject diagnosed with Non-small cell lung carcinoma; and
b. Determining the subject's disease monitoring or progression based on determination carried out in step (a),

wherein the upregulation of the levels of ITIH1, LOXL2, GELSOLIN, FBN2, or any combination thereof, isolated or present inside the circulating exosomes is indicative of a poor overall survival or of a poor PFS of the subject.

It is further noted that for monitoring the progression of the disease of the sixth aspect of the invention, or of any preferred embodiments therefrom, such determination is made by comparing the levels of ITIH1, LOXL2, GELSOLIN, FBN2, or any combination thereof, with a threshold or cutoff level, wherein preferably such level is obtained from a group of healthy controls or corresponds to the mean value of intensity of each protein (88006.8; 5733630.8; 352569 and 355354.2 Arbitrary Unit of Intensity, respectively) in NSCLC patients.

A seventh aspect of the invention refers to a method for predicting the response to treatment of a subject treated with a drug selected from the list consisting of: radiotherapy, immunotherapy, chemotherapy, DNA alkylating agent, DNA cross-linking agent, or any combination thereof, wherein such subject is diagnosed with NSCLC, and wherein such method comprises the following steps:
a. Performing the methodology according to any of the fourth or sixth aspects of the invention; and
b. Determining the response to treatment of the subject's disease based on the upregulation of any of the biomarkers, or combinations thereof identified in any of the fourth or sixth aspects of the invention.

In this sense, a higher level of any of the biomarkers, or combinations thereof, identified in any of the fourth or sixth aspects of the invention in comparison to a sample obtained from the subject previously or with respect to a reference value, is indicative of an unfavourable response to treatment.

It is noted that with respect to the seventh aspect of the invention, if the intent is to determine patient's response to treatment before the onset of treatment, a higher level of any of the biomarkers, or combinations thereof, identified in any of the fourth or sixth aspects of the invention with respect to a healthy control subject or with respect to a reference value is indicative of a potentially non favorable disease response to treatment with an alkylating agent, radiation therapy, immunotherapy, chemotherapy, or any combination thereof; and wherein once treatment has commenced the gradual decrease in the level is indicative of a favorable tumor response to treatment. Preferably, said alkylating agent is selected from the list consisting of nitrogen mustards such as mechlorethamine, cyclophosphamide, ifosfamide, melphalan, or chlorambucil; ethyleneimines and methylmelamines such as altretamine; methylhydrazine derivatives such as procarbazine; alkyl sulfonates such as busulfan; nitrosoureas such as carmustine; triazenes such as dacarbazine or temozolomide; or platinum coordination complexes such as cisplatin, carboplatin, oxaliplatin, dicycloplatin, eptaplatin, lobaplatin, Miriplatin, Nedaplatin, Oxaliplatin, Picoplatin, Satraplatin, Triplatin or tetranitrate. Such determination of the response to treatment is performed on the basis of the prediction of the progression-free survival or overall survival of the subject.

An eight aspect of the invention refers to a kit comprising the reagents necessary to implement the use of any of the first to third aspects of the invention, or the method of any of the fourth to seventh aspects of the invention. Preferably, such reagents comprise specific primers for determining the levels of miR-142, miR-451a, or any combination thereof, or the levels of miRNA-55745. More preferably, the specific Taqman probes (Thermofisher Scientific) for determining the levels of miR-142, miR-451a, or any combination thereof are: 477910_mir and 478107_mir, respectively. The miRbase accession numbers of miR-142-3p and miR-451a are: MI0000458 and MI0001729, respectively. Also preferably, the specific Taqman probe for determining the levels of miRNA-55745 is a customized probe developed with the mature sequence: agugaaaugacuugagagg.

Alternatively to the eight aspect of the invention, the present invention refers to a kit comprising the reagents for determining the levels of ITIH1, LOXL2, GELSOLIN, FBN2, or any combination thereof.

A ninth aspect of the invention refers to the use of the kit of the eight aspect of the invention to implement the use of any of the first to third aspects of the invention, or the method of any of the fourth to seventh aspects of the invention.

As used herein, “means for detecting or determining the levels of a miRNA” is understood as labeled complementary DNA, RNA, modified nucleic acid strand (i.e. probe) to localize a miRNA sequence in a biological sample such as a blood, serum or plasma sample or in a portion or section of tissue, in the entire tissue or cells.

A tenth aspect of the invention relates to a nucleotide sequence consisting of SEQ ID NO 1. This aspect also refers to means for detecting or determining the levels of SEQ ID NO 1.

An eleventh aspect of the invention refers to a fully complementary (100% complementary) DNA, RNA or modified nucleic acid strand (i.e. probe) capable of hybridizing with miRNA sequence SEQ ID NO 1, in particular with no mismatches.

A preferred embodiment of the prior aspects of the invention refers to a genetic construct, such as a DNA sequence, capable of transcribing miRNA sequence SEQ ID NO 1.

Another preferred embodiment refers to a kit or device comprising the means for detecting or determining the levels of SEQ ID NO 1.

The following examples help to illustrate but do not limit the present invention.

Methods to Determine the Amount of a miRNA

Many methods of quantifying miRNAs are contemplated. Any reliable, sensitive, and specific method for quantifying miRNAs within exosomes can be used in the present invention. In some embodiments provided, a target miRNA or the reference miRNA is preferably amplified prior to or during quantification. In other embodiments, the miRNA is not amplified as part of the quantification process.

Amplification Reactions

Many methods exist for amplifying miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain preferred embodiments, more than one amplification method is used, such as reverse transcription followed by real time PCR (Chen et al., Nucleic Acids Research, 33(20):e179, (2005)).

A typical PCR reaction includes multiple amplification steps, or cycles that selectively amplify target nucleic acid species. A typical PCR reaction includes three steps: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single-stranded, a reverse transcription reaction (which produces a complementary cDNA sequence) is performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depend on many factors, including, but not limited to, the desired hybridization temperature between the primers, the target nucleic acid sequence, and the complexity of the different target nucleic acid sequences to be amplified. In preferred embodiments, a primer is about 15 to about 35 nucleotides in length. In other preferred embodiments, a primer is equal to or fewer than 15, 20, 25, 30, or 35 nucleotides in length. In additional preferred embodiments, a primer is at least 35 nucleotides in length.

In preferred embodiments of the invention, forward primers can comprise at least one sequence that anneals to a target miRNA and/or to the reference miRNA and alternatively can comprise an additional 5′ non-complementary region. In another embodiment, reverse primers can be designed to anneal to the complement of a reverse transcribed miRNA. The reverse primer may be independent of the target miRNA or reference miRNA sequence, and multiple target miRNAs and the reference miRNAs may be amplified using the same reverse primer. Alternatively, a reverse primer may be specific for a target miRNA and for the reference miRNA.

In some preferred embodiments, two or more miRNAs are amplified in a single reaction volume (one or more target miRNAs and the reference miRNA, for example). Normalization may alternatively be performed in separate reaction volumes. One preferred embodiment includes multiplex q-PCR, such as qRT-PCR, which enables simultaneous amplification and quantification of at least one miRNA of interest and at least the reference miRNA miR-151a-3p in one reaction volume by using more than one pair of primers and/or more than one probe. The primer pairs may comprise at least one amplification primer that uniquely binds each miRNA, and the probes are preferably labelled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

A single combined reaction for q-PCR, is desirable for several reasons: (1) decreased risk of experimenter error, (2) reduction in assay-to-assay variability, (3) decreased risk of target or product contamination, and (4) increased assay speed. The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

Detection of miRNAs

In preferred embodiments, labels, dyes, or labelled probes and/or primers are used to detect amplified or unamplified miRNAs. Depending on the sensitivity of the detection method and the abundance of the target, for example, amplification may or may not be required prior to detection. One skilled in the art will recognize the detection methods where miRNA amplification is preferred.

A probe or primer may include Watson-Crick bases or modified bases. Modified bases include, but are not limited to, the AEGIS bases (from Eragen Biosciences), which have been described, e.g., in U.S. Pat. Nos. 5,432,272, 5,965,364, and 6,001,983. In certain preferred embodiments, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a preferred embodiment, oligonucleotide probes or primers present in a multiplex amplification are suitable for monitoring the amount of amplification product produced as a function of time. In certain preferred embodiments, probes having different single stranded versus double stranded character are used to detect the nucleic acid. Probes include, but are not limited to, the 5′-exonuclease assay (e.g., TaqMan™) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™ probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain preferred embodiments, one or more of the primers in an amplification reaction can include a label. In yet further embodiments, different probes or primers comprise detectable labels that are distinguishable from one another. In some preferred embodiments a nucleic acid, such as the probe or primer, may be labelled with two or more distinguishable labels.

In preferred embodiments, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other embodiments, use of labels can be accomplished using any one of a large number of known techniques employing known labels, linkages, linking groups, reagents, reaction conditions, and analysis and purification methods.

miRNAs can be detected by direct or indirect methods. In a direct detection method, one or more miRNAs are detected in the exosomes by a detectable label that is linked to a nucleic acid molecule. In such methods, the miRNAs may be labelled prior to binding to the probe. Therefore, binding is detected by screening for the labelled miRNA that is bound to the probe. The probe is optionally linked to a bead in the reaction volume.

In certain preferred embodiments, nucleic acids are detected in the exosomes by direct binding with a labelled probe, and the probe is subsequently detected. In one preferred embodiment of the invention, the nucleic acids, such as amplified miRNAs, are detected using FlexMAP Microspheres (Luminex) conjugated with probes to capture the desired nucleic acids. Some methods may involve detection with polynucleotide probes modified with fluorescent labels or branched DNA (bDNA) detection, for example.

In other preferred embodiments, nucleic acids are detected in the exosomes by indirect detection methods. In such an embodiment, it is preferred that a biotinylated probe is combined with a streptavidin-conjugated dye to detect the bound nucleic acid. The streptavidin molecule binds a biotin label on amplified miRNA, and the bound miRNA is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniquies, Academic Press, San Diego (1992) and Garman A., Non-Radioactive Labeling, Academic Press (1997).). Fluorescent reporter dyes useful as labels include, but are not limited to, fluoresceins (see, e.g., U.S. Pat. Nos. 5,188,934, 6,008,379, and 6,020,481), rhodamines (see, e.g., U.S. Pat. Nos. 5,366,860, 5,847,162, 5,936,087, 6,051,719, and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, FluorX (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain preferred embodiments, the fluorescent label is selected from SYBR-Green, 6-carboxyfluorescein (“FAM”), TET, ROX, VIC™, and JOE. For example, in certain preferred embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some preferred embodiments. It will be appreciated that pairs of fluorophores are chosen that have distinct emission spectra so that they can be easily distinguished.

In still a further preferred embodiment, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further preferred embodiments, methods relying on hybridization and/or ligation to quantify miRNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 (incorporated herein by reference) may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify unhybridized HARP probe such that it cannot be amplified.

In an additional preferred embodiment of the method, a probe ligation reaction may be used to quantify miRNAs. In a Multiplex Ligation-dependent Probe Amplification (MLPA) technique (Schouten et al., Nucleic Acids Research 30:e57 (2002)) pairs of probes which hybridize immediately adjacent to each other on the target nucleic acid are ligated to each other only in the presence of the target nucleic acid. In some preferred embodiments, MLPA probes have flanking PCR primer binding sites. MLPA probes can only be amplified if they have been ligated, thus allowing for detection and quantification of target miRNA or reference miRNA.

II. Normalization

Methods of normalization and kits for exosome cargo normalization are provided herein. The methods correct for exosome sample-to-exosome sample variability by comparing a target measurement in a sample to the reference miRNA control presented herein. Normalization of miRNA quantification assays reduces systematic (non-biological) and non-systematic differences between samples, and is critical for accurate measurement of differential miRNA content, for example.

The accurate measurement of the biological differential content between two groups of exosomes samples (or samples comprising exosomes or exosome content) is the goal of many miRNA qRT-PCR assays. Yet, miRNA levels in qRT-PCR reactions can vary from one exosomes samples (or samples comprising exosomes or exosome content) to the next for reasons that may be technical or biological.

Technical reasons may include variabilities in tissue procurement or storage, inconsistencies in RNA extraction or quantification, or differences in the efficiency of the reverse transcription and/or PCR steps. Biological reasons may include exosomes samples (or samples comprising exosomes or exosome content)-to-sample heterogeneity in cellular populations, differences in bulk transcriptional activity, or alterations in specific miRNA content that is linked to an aberrant biological program (e.g., a disease state). Given the multiplicity of sources that can contribute to differences in miRNA quantification, results from qRT-PCR assays should be normalized against a relevant endogenous target or targets to minimize controllable variation, and permit definitive interpretations of nominal differences in exosome cargo normalization.

Preferred embodiments comprise multiplex methods for quantifying and normalizing the amount of a target miRNA in a biological sample. In a preferred embodiment of the invention, the amount of one or more target miRNAs is measured in the exosomes in a reaction volume, and the amount of at least the reference miRNA miR-151a-3p measured in the reaction volume. The amount of target miRNA is normalized based on the amount of the reference miRNA.

For experiments using only the reference miRNA miR-151a-3p for normalization, the data are normalized to the measured quantity of said one reference miRNA. When two or more reference miRNAs are used as normalizers, a mean of the normalizers (e.g. arithmetic mean or geometric mean) is preferably used, depending on the nature of the quantification data. For example, the threshold cycle (Ct) values obtained from q-PCR experiments may be normalized to the geometric mean of two or more normalizers. Data represented on a linear scale (absolute levels data) may be normalized to an arithmetic mean of normalizers. Additional methods of combining normalizers are also contemplated, such as weighted averages.

In some embodiments, levels may be normalized using a comparative Ct method for relative quantification between samples or sample types. The general methods for conducting such assays are described, e.g., in Real-Time PCR Systems: Applied Biosystems 7900HT Fast Real-Time PCR System, and 7300/7500 Real-Time PCR Systems, Chemistry Guide, Applied Biosystems, 2005, Part No. 4348358.

Many additional methods of normalization are well known to those skilled in the art, and all normalization methods are contemplated. Those skilled in the art will recognize the appropriate normalization methods for each quantification and detection method described herein.

III. Reference MiRNA miR-151a-3p

Preferred embodiments of the invention include measuring the amount of at least miR-151a-3p reference miRNA in the exosomes contained in a biological sample, more particularly in a biological sample containing circulating exosomes or tissue exosomes miRNAs, and normalizing the amount of a target miRNA to the amount of said reference miRNA. In this sense, miR-151a-3p is understood herein to be stably expressed in the exosomes from any human origin, and does not show significant differential content in the exosomes from healthy or diseased individuals. Therefore, in the normalization methods provided herein, the amount of target miRNA in the exosomes of any biological sample of human origin can be normalized to the amount of at least miR-151a-3p as a reference miRNA in the exosomes from the biological sample.

A “biological sample” is any sample or specimen derived from a human that contains exosomes. For example, the biological sample may be a patient sample. A “patient sample” is any biological specimen from a patient. The term includes, but is not limited to, biological fluids such as blood, serum, plasma, urine, cerebrospinal fluid, tears, saliva, lymph, dialysis fluid, lavage fluid, semen, and other liquid samples, as well as cells and tissues of biological origin. The term also includes cells isolated from a human or cells derived therefrom, including cells in culture, cell supernatants, and cell lysates. It further includes organ or tissue culture-derived fluids, tissue biopsy samples, tumor biopsy samples, stool samples, and fluids extracted from physiological tissues, as well as cells dissociated from solid tissues, tissue sections, and cell lysates. A biological sample may be obtained or derived from tissue types including but not limited to lung, liver, placenta, bladder, brain, heart, colon, thymus, ovary, adipose, stomach, prostate, uterus, skin, muscle, cartilage, breast, spleen, pancreas, kidney, eye, bone, intestine, esophagus, lymph nodes and glands. The term “biological sample” encompasses samples that have been manipulated in any way after their procurement, such as by treatment with preservatives, cellular disruption agents (e.g. lysing agents), solubilization, purification, or enrichment for certain components, such as polynucleotides, in certain aspects. Also, derivatives and fractions of patient samples are included. A sample may be obtained or derived from a patient having, suspected of having, or recovering from a disease or pathological condition. Diseases and pathological conditions include, but are not limited to, proliferative, inflammatory, immune, metabolic, infectious, and ischemic diseases. Diseases (e.g. cancers) also include neural, immune system, muscular, reproductive, gastrointestinal, pulmonary, cardiovascular, and renal diseases, disorders, and conditions. In a preferred embodiment said biological sample comprises serum, whole blood or platelets

The following examples are merely illustrative of the present invention.

### Examples

Material and Methods

Cell Culture Lung and ovarian cancer cell lines H23, H460, H1299, H727, A2780 and OVCAR3 were purchased from the ATCC (Manassas, Va.) or ECACC (Sigma-Aldrich, Spain). All of them were maintained in RPMI supplemented with 10% exosome-depleted FBS, except for OVCAR3 which was cultured in RPMI with 20% exosome-depleted FBS. FBS was depleted of bovine exosomes by ultracentrifugation at 100,000×g for 16 h at 4° C. The CDDP-resistant variants H23R, H460R, A2780R and OVCAR3R, and the carboplatin-resistant variants H23R-CBDCA and A2780R-CBDCA were established by exposing cells to increasing doses of each platinum-based drug as described. The CDDP sensitive and resistant ovarian cancer cell lines 41M and 41MR were provided by Dr. Kelland (UK) and maintained in DMEM supplemented with 10% exosome-depleted FBS.

NSCLC Clinical Samples and Data Collection

Plasma samples from 51 advanced NSCLC patients (stages IIIA to IV) from La Paz University Hospital were collected before received any platinum-based treatment. We also collected 10 plasma samples from healthy donors. Follow-up was conducted according to the criteria of the medical oncology division of La Paz University Hospital. All the samples were processed following the standard operating procedures with the appropriate approval of the Human Research Ethics Committees, including informed consent within the context of research. Clinical, pathological and therapeutic data were recorded by an independent observer and blinded for statistical analysis.

Exosome Isolation

Cell line-derived exosomes, used for functional viability assays, were isolated from 500 ml of supernatant collected after 48-72 h of cell sowing. Supernatant debris was pelleted by centrifugation at 1500×g for 30 min. The supernatant was concentrated until obtaining 50 ml of each cell line using Ultra-15 Centrifugal Filter Concentrator (Merck, Germany) and filtrated with 0.2 μm filter to eliminate larger vesicles. Exosomes were then harvested by centrifugation at 100,000×g for 2 h. The exosome pellet was resuspended in 35 ml of 0.2 μm-filtered 1×PBS and collected by ultracentrifugation at 100,000×g for 2 h (45ti rotor, Optima L-100 XP Centrifuge, Beckman Coulter, USA). Exosome pellets were resuspended in 200-300 μL of 0.2 μm-filtered 1×PBS and stored at −80° C. Cell line-derived exosomes, used for small RNAseq and proteomic analysis, were isolated by miRCURY Exosome Isolation Kit (Exiqon, Denmark) according to manufacturer's instructions. Circulating exosomes and their miRNA content from plasma samples were obtained with exoRNeasy Serum/Plasma Midi Kit (Qiagen, Germany). Circulating exosomes from plasma samples used for proteomic analysis were isolated with qEV original Size Exclusion Column (iZON Science, UK).

Exosome Quantitation and Size Determination

Size and concentration of isolated exosome samples were characterized using the LM10 nanoparticle characterization system NanoSight (Malvern, UK). Samples were diluted from 1:400 to 1:100 in 0.2 μm-filtered 1×PBS, depending on the concentration. Each sample analysis was conducted 3 times for 30 seconds each time. The Nanosight automatic analysis settings were used to process the data.

Electron Microscopy

Exosomes from resistant cells were purified as described above and fixed in 100 μl of 2% PFA 0.1M phosphate buffer (pH 7.4).

Exosome Labelling and Uptake Assays by Flow Cytometry

Ultracentrifuge-obtained exosomes from culture medium of resistant cell lines were fluorescently labeled using PKH26 Red Fluorescent Cell Linker Mini Kit (Merk, Germany) according to the manufacturer's protocol. Briefly, 250 μl of Dilutent C mixed with 1 μl of PKH26 were prepared for each sample. Exosome pellets were blended with the stain solution and incubated for four minutes. The labelling reaction was stopped by adding an equal volume of 3% BSA 0.2 μm-filtered 1×PBS. Labelled exosomes were washed in 35 ml of 3% BSA 0.2 μm-filtered 1×PBS, collected by ultracentrifugation at 100,000×g for 2 h and resuspended in PBS. Exactly the same process was performed with PBS as control to determine our PKH26 background.

For the uptake assays, sensitive cells were labelled with CellTrace Violet (CTV). Briefly, 10⁶ cells resuspended in PBS+5% exosome-depleted FBS were incubated 20 min with 20 μL of 1:100 CTV dilution. After washing the excess of dye with culture medium, cells were co-seeded with exosomes or PBS labelled with PKH26. After 20 h of co-incubation, we trypsinized, washed and passed cells through the flow cytometer. To measure the death rate associated with the incubation with exosomes, we stained cells with 7-Aminoactinomycin D (7AAD).

Functional Assays

41M and 41MR were seeded in p96 multiwell plates in a concentration of 20000 cells/well. Exosomes isolated as described above were quantified by Bradford assay (Bio-rad, CA, USA) and co-incubated with sensitive cells in a concentration of 0.0008 μg of exosomes per cell for 24, 48 and 72 h. After the incubation each case, cells were treated with increasing doses of cisplatin (Farma-Ferrer, Barcelona, Spain). Cell viability was measured 72 h after the drug treatment using MTS assay. H23, H460 and A2780 assays were performed the same as described with 41M and 41MR but co-incubating cells with exosomes for 48 h and using two different concentrations of exosomes: 80 μg/ml and 800 μg/ml.

Small RNAseq

RNA from cell line exosomes was extracted using miRCURY RNA Isolation Kit (Exiqon, Denmark).

qRTPCR

RNA from cell line exosomes and from plasma samples was extracted as described above. Total miRNAs were retrotranscribed using TaqMan™ Advanced miRNA cDNA Synthesis Kit (Thermofisher Scientific). Quantitative RT-PCR analysis was performed using TaqMan™ Advanced miRNA assays (Thermofisher Scientific) and TaqMan™ Universal PCR Master Mix (Applied Biosystems, Spain) or TaqMan™ Fast Advanced Master Mix, depending on the assay. Samples were analysed in triplicate using the HT7900Real-Time PCR System (Applied Biosystems, USA) and relative gene expression or level quantification was calculated according to the comparative threshold cycle method 2^{-ΔCt}, where ΔCt were calculated by subtracting the Ct value of the endogenous control miR-151a from the Ct value of the targeted miRNA.

Proteomic Analysis

Samples were previously quantified by microBCA analysis (Pierce) and similar amounts (1.5 μg per sample) were individually dissolved in 8M urea, 25 mM ammonium bicarbonate, reduced with DTT and alkylated with iodoacetamide, according to a method previously described. Urea concentration was reduced to 2M with 25 mM ammonium bicarbonate (final volume 40 μL) and the samples digested overnight at 37° C. with recombinant MS-grade trypsin (Sigma-Aldrich), with a ratio of 25:1. After digestion, samples were desalted using ZipTip (Merck) as described. Synthetic peptides were used to validate MRM methods or to confirm the peptide sequence by shotgun proteomics. Candidate peptides were synthesized using standard Fmoc chemistry in an Intavis Multiple peptide synthesizer (INTAVIS, Cologne, Germany). Digested samples were diluted with 0.2% TFA in water and subjected to MRM analysis using a 1D Plus nanoLC Ultra system (Eksigent, Dublin, Calif., USA) interfaced to a Sciex 5500 QTRAP triple quadrupole mass spectrometer (Sciex, Framingham, Mass., USA) equipped with a nano-ESI source and controlled by Analyst v.1.5.2. software (ABSciex). Tryptically digested samples were loaded online on a C18 PepMap 300 μm I.D.×5 mm trapping column (5 μm, 100 Å, Thermo Scientific) and separated using a BioSphere C18 75 μm i.d.×150 mm capillary column (3 μm, 120 Å, Nanoseparations). Gradient elution was performed according the following scheme: isocratic conditions of 98% A (water containing 0.1% formic acid): 2% B (100% ACN with 0.1% formic acid) for two minutes, a linear increase to 40% B in 45 min, a linear increase to 95% B in one min, isocratic conditions of 95% B for five minutes and return to initial conditions in five minutes. Injection volume was 5 μL. The liquid chromatographic system was coupled via a nanospray source to the mass spectrometer. Experimental settings for MRM analysis initially were set up with the help of Skyline v.3.6 software (see citation). A list of transitions (usually 3-4 per peptide, with a preference toward higher-mass y series ions) as well as collision energy values were determined automatically for the candidate peptides. Initial experiments were performed at a scan time (dwell time) of 20 ms for 100-1250 m/z mass range. Chromatographic retention time values obtained for the list of peptides were used to create a scheduled version of the MRM method. The MS analysis was conducted in the positive ion mode with the ion spray voltage set at 2800 V. Drying gas temperature was set to 150° C. at a flow rate of 20 L/min. Peak areas and signal-to-noise (S/N) values for each transition were determined using Skyline v.3.6 20.

For some representative samples shotgun proteomics analysis was performed as follows: nano LC ESI-MSMS analysis was performed using an Eksigent 1D-nanoHPLC coupled to a 5600TripleTOF QTOF mass spectrometer (Sciex, Framinghan, Mass., USA). The analytical column used was a silica-based reversed phase column Waters nanoACQUITY UPLC 75 μm×15 cm, 1.7 μm particle size. The trap column was an Acclaim PepMap 100, 5 μm particle diameter, 100 Å pore size, switched on-line with the analytical column. The loading pump delivered a solution of 0.1% formic acid in 98% water/2% acetonitrile (Scharlab, Barcelona, Spain) at 3 μL/min. The nanopump provided a flow-rate of 250 nL/min and was operated under gradient elution conditions, using 0.1% formic acid (Fluka, Buchs, Switzerland) in water as mobile phase A, and 0.1% formic acid in 100% acetonitrile as mobile phase B. Gradient elution was performed according the following scheme: isocratic conditions of 96% A: 4% B for five minutes, a linear increase to 40% B in 25 min, a linear increase to 95% B in two minutes, isocratic conditions of 95% B for five minutes and return to initial conditions in 10 min. Injection volume was 5 μL. The LC system was coupled via a nanospray source to the mass spectrometer. Automatic data-dependent acquisition using dynamic exclusion allowed obtaining both full scan (m/z 350-1250) MS spectra followed by tandem MS CID spectra of the 15 most abundant ions. Acquisition time was 250 ms and 100 ms for MS and MSMS spectra, respectively.

Cell Transfection

Each cell line was seeded in p24 multiwell plates for the platinum-viability assays, one million of cells each one. The day after, cells were transfected with 10 or 20 nM of the specific mimic assay or negative control (Thermofisher) and using JetPrime according to the manufacturer's protocol. After 6 h of the transfection, cells were treated with increasing doses of cisplatin or carboplatin (Farma-Ferrer, Barcelona, Spain). For each cell line, the platinum concentration was calculated given their IC50. Cells were stained 72 h after the drug treatment by glutaraldehyde 1% and crystal violet 0.1%. After that, cells were treated with acetic acid 10% to fade them and measure the absorbance with Infinite 200 PRO multimode reader (TECAN). miRNA overexpression was measured from P35 plates that were transfected at the same time of p24 multiwell plates and were stored at −80° C. until the RNA extraction for qRT-PCR analysis.

Results

Exosomes from Cisplatin-Resistant Cells Confer Drug Resistance to Sensitive Cells CDDP-Resistant-Exosomes Alter the Drug Response of Sensitive Cells

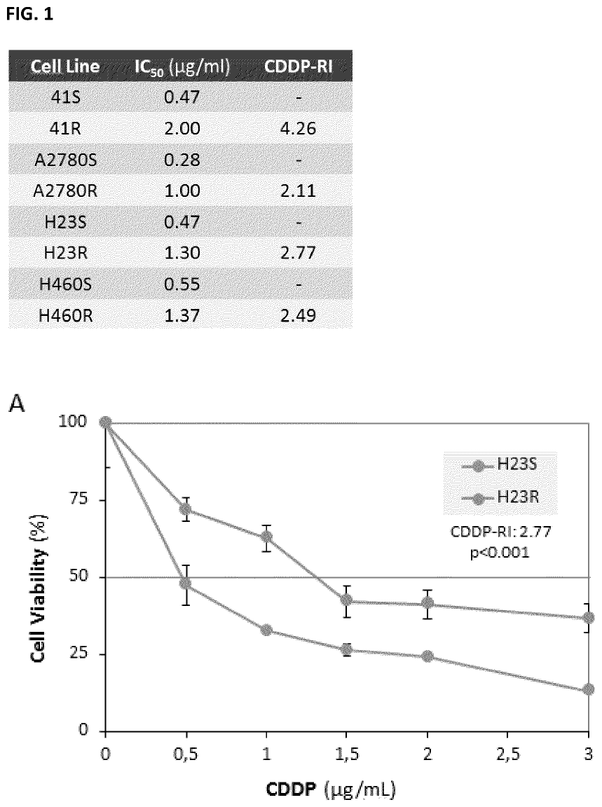

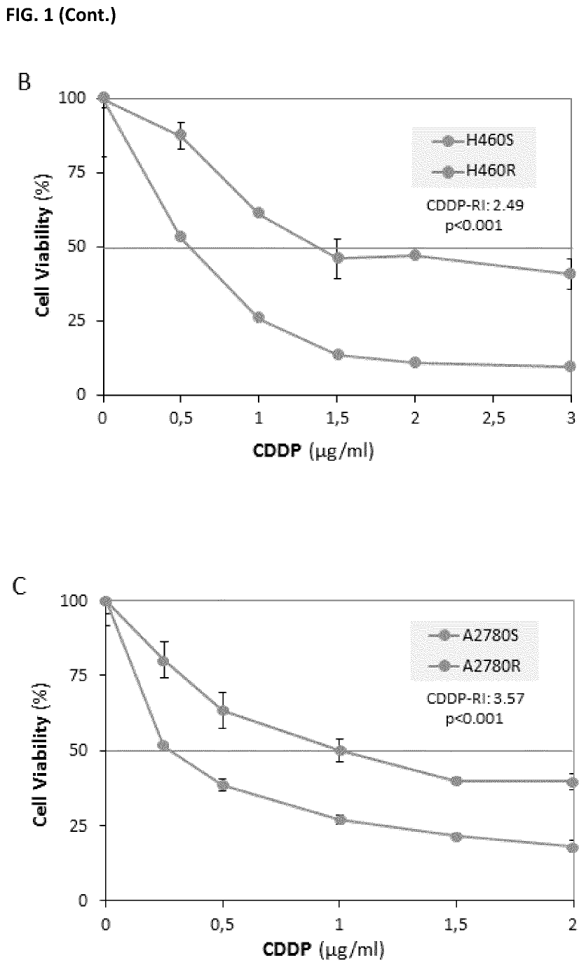

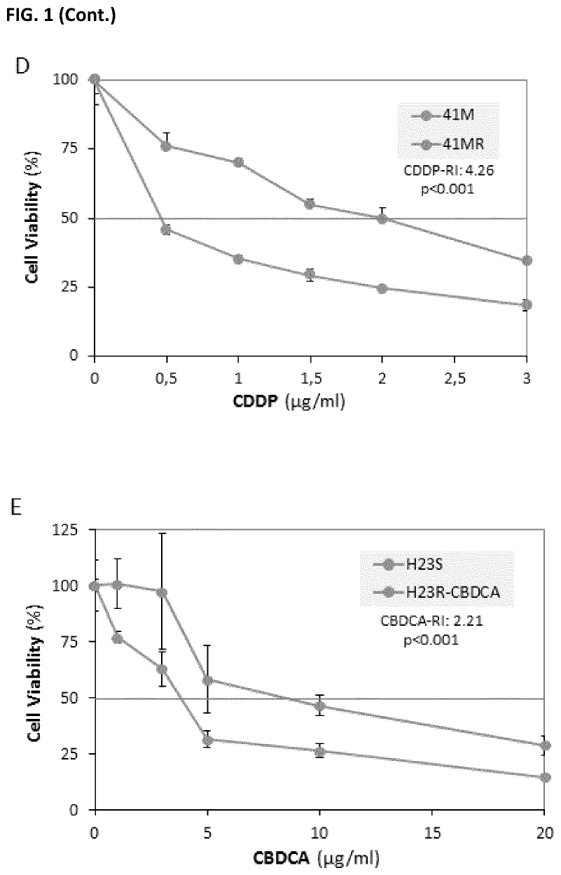

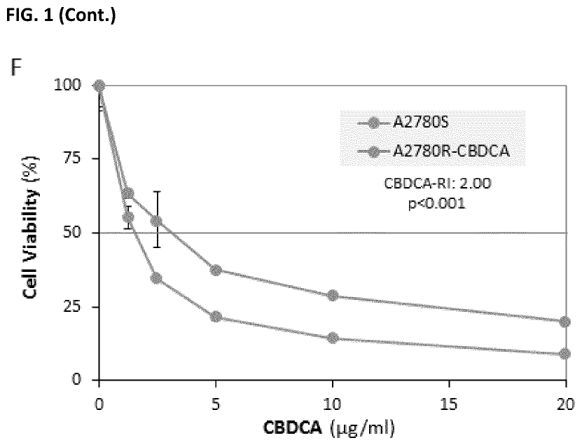

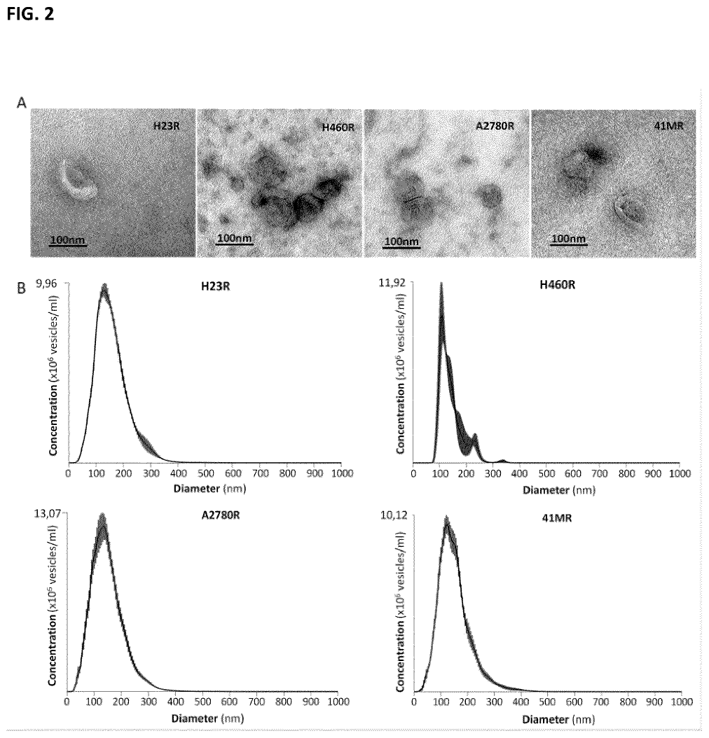

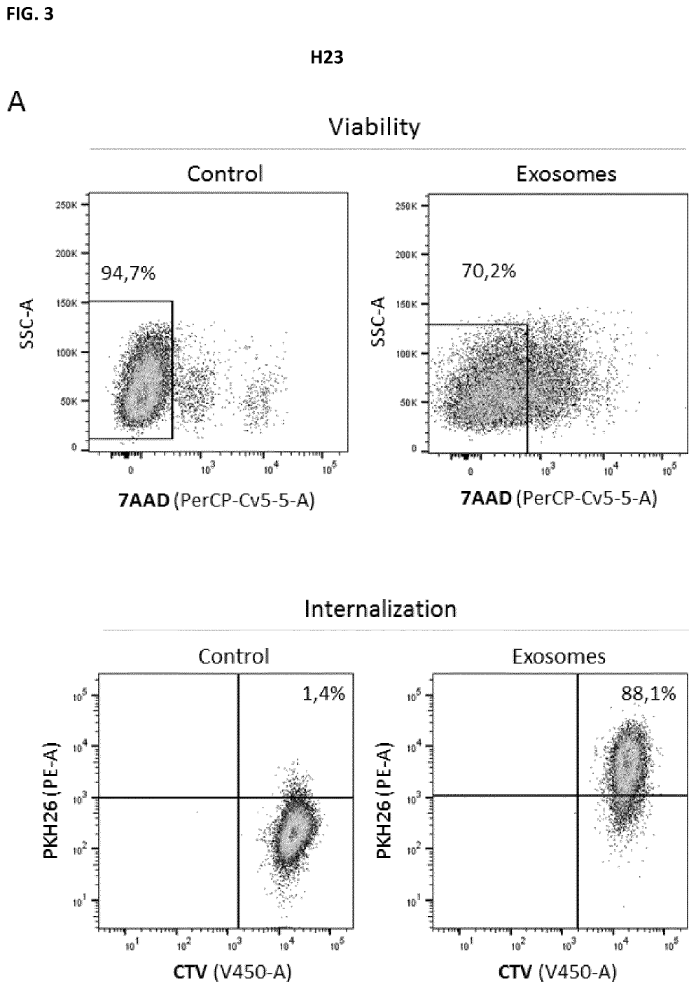

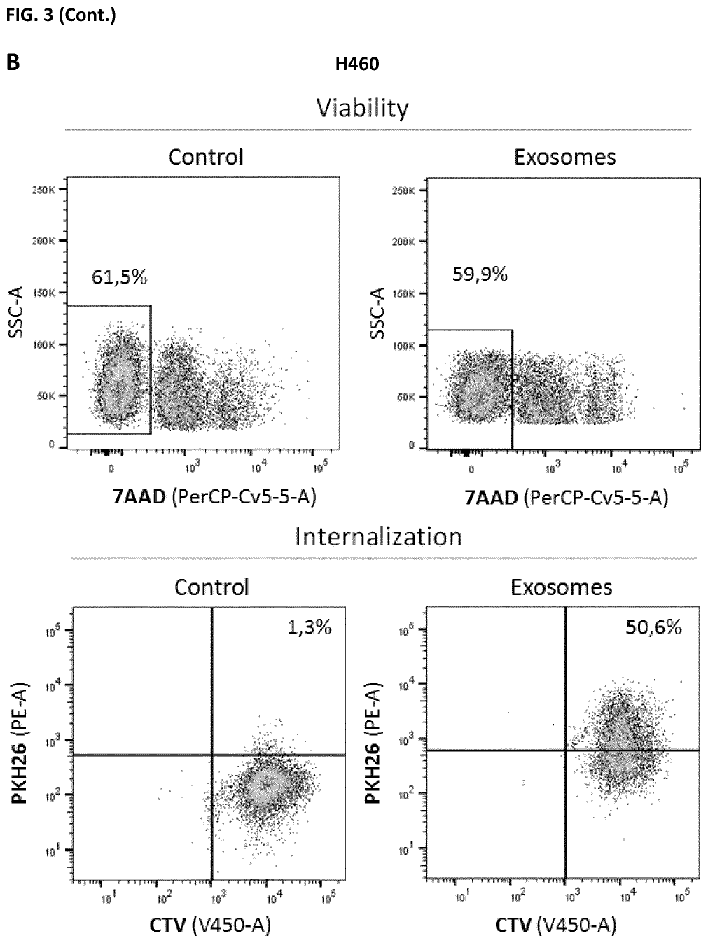

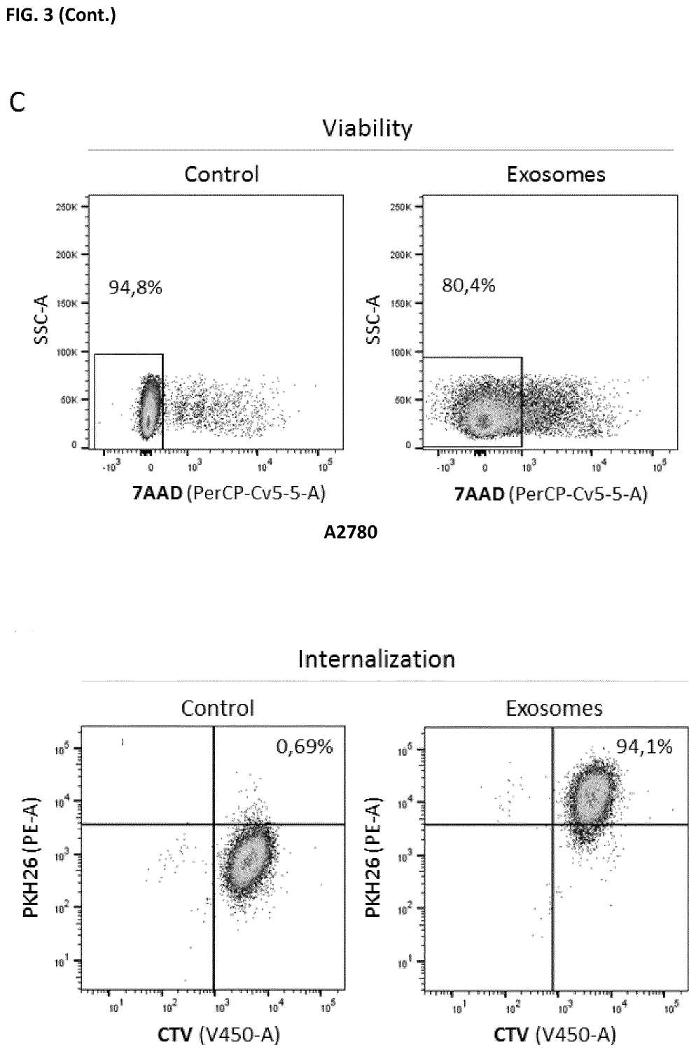

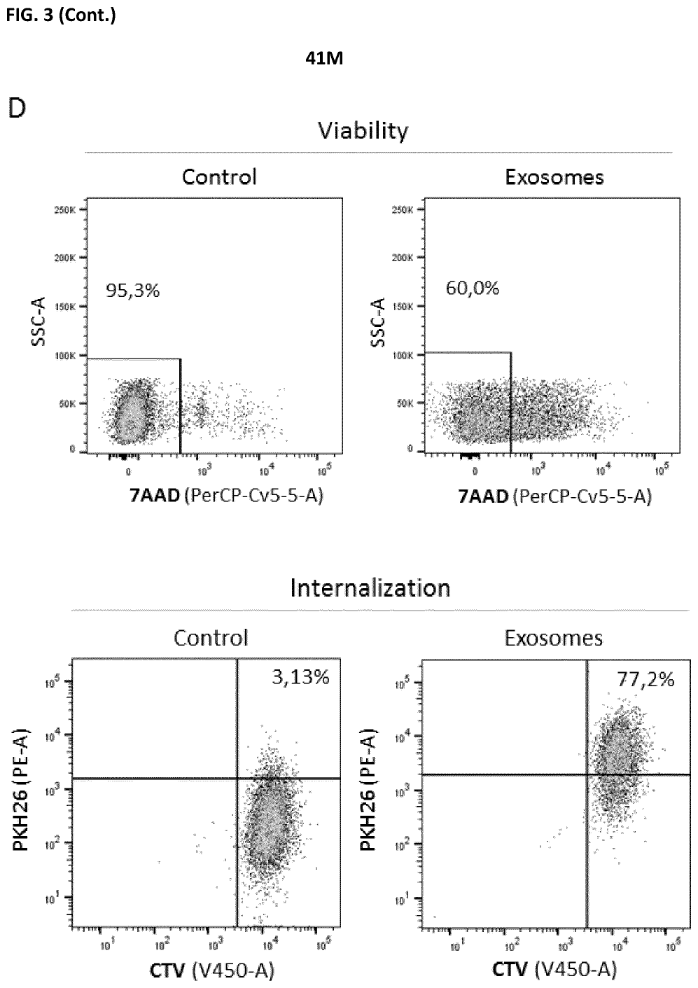

To determine whether cisplatin-resistant cells transmit their resistance through exosomes we used matched CDDP-sensitive/resistant lung cancer and ovarian cell lines H23S/H23R, H460S/H460R, A2780S/A2780R and 41M/41MR and, that we established previously (Ibanez de Caceres, I. et al. IGFBP-3 hypermethylation-derived deficiency mediates cisplatin resistance in non-small-cell lung cancer. Oncogene 29, 1681-1690 (2010); Vera, O. et al. DNA Methylation of miR-7 is a Mechanism Involved in Platinum Response through MAFG Overexpression in Cancer Cells. Theranostics 7, 4118-4134 (2017)). First, we confirmed that resistant subtypes showed, at least, two times more drug resistance than the paired parental cell line (Respectively 2.77, 2.49, 2.11 and 4.26 of Resistant-Index (RI); p<0.001) (FIG. 1). We then isolated exosomes from resistant subtypes H23R, H460R, 41MR and A2780R and confirmed their proper size (around 100 nm) using electron microscopy and NanoSight analysis (FIGS. 2A and B) Once we have confirmed that our samples were successfully enriched in exosomes, we wanted to assess if exosomes from cisplatin-resistant cells could alter the drug response of sensitive cells. For that purpose, firstly, we examined whether exosomes isolated from CDDP resistant subtypes were taken up by sensitive parental cells, we labelled exosomes with PKH26 and exposed them to sensitive cells. The analysis using flow cytometry confirmed the uptake of resistant exosomes in more than 77% of sensitive cells H23S, 41M and A2780S compared with PBS control (FIG. 3).

After confirming that internalization of the resistant exosomes occurred in more than 75% of the H23S, A2780S and 41M cells, and in 50% of the H460S cells, we evaluated whether said resistant exosomes were capable of altering the pharmacological response of the sensitive cells.

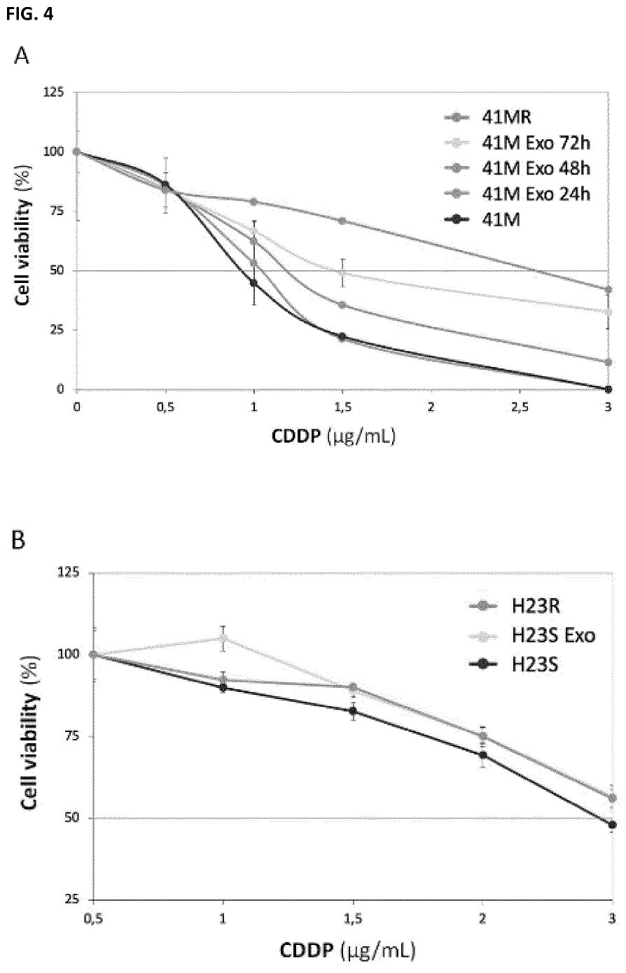

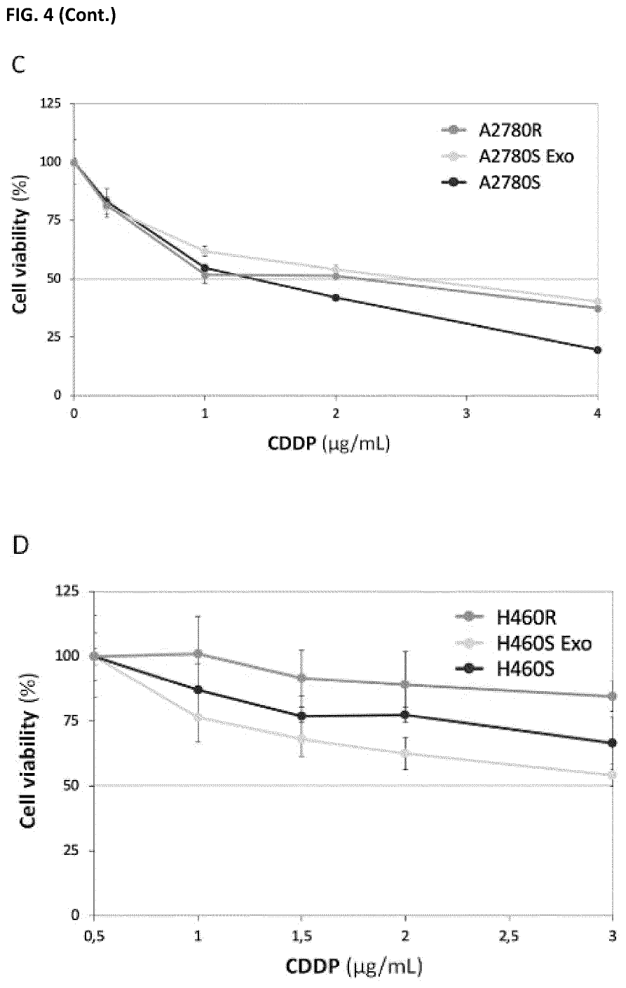

We co-incubated 41M parental cell line with exosomes from 41MR resistant-cells during 24, 48 and 72 h before treating them with increasing doses of cisplatin. Results showed a significant decrease in the response of the cells to the drug in a time-of-incubation-dependent manner, reaching the highest resistance with 72 h of incubation (FIG. 4A).

We then tested the viability to cisplatin of H23S, H460S and A2780S after 72 h of incubation with the exosomes from their CDDP-resistant cell subtype each case. H23S and A2780S increased their resistance to cisplatin when they were incubated with the exosomes, reaching a viability similar to the resistant cells (FIGS. 4B and C). However, H460S showed no change in response to the drug (FIG. 4D).

Characterization of the Exosome Content in Resistant Phenotypes

To further investigate the molecular mechanisms behind the acquisition of resistance in H23, A2780 and 41 sensitive cells through CDDP-resistant exosomes, we characterized their miRNA and protein content. We wanted to focus on these active molecules since they are able to generate post-translational changes themselves. Firstly, we performed a small RNA-seq on total RNA extracted from exosomes of the three matched CDDP-sensitive and resistant cell lines. We wanted to compare their miRNA profile in order to identify candidates overrepresented in resistant exosomes that potentially induce the resistance in parental cells. After the bioinformatic processing for the normalization of the results of each pair of lines in the unit of reference of massive sequencing “transcripts per million readings” (TPM), 603 and 506 miRNAs were identified in H23S and H23R, 754 and 750 in A2780S and A2780R; 725 and 790 in 41M (also called 41S) and 41MR (also called 41R) present in the exosomal content of the secretome, respectively. Firstly, we filtered the data choosing common miRNAs in both subtypes for each pair of cell lines, in order to discard those miRNAs in which there could be errors in the capture. After that we obtained that 355 miRNAs were selected in H23, 460 in A2780 and 499 in 41 cell line. Then, we selected miRNAs overrepresented in resistant exosomes in comparison with the corresponding sensitive ones with a difference of more than 4 times (Fold Change >2): 16 in H23, 2 in A2780 and 30 miRNAs in 41

To increase the success rate of our screening and find statistically robust candidates for subsequent clinical uses, we selected those miRNAs shared at least for two of the three lines: miR-892a, miR-891a-5p, miR-451a, miR-363-3p and miR-142-3p (FIG. 5, left panel). In parallel, the bioinformatic analysis of the small RNA-seq data allowed us to identify new miRNAs, not previously described in any current database (unknown miRNAs). We followed the same selection workflow as with the known miRNAs, so after the TPM normalization 356 and 254 miRNAs were identified in H23S and H23R, 335 and 461 miRNAs in A2780S and A2780R, and 325 and 383 in 41S and R, respectively. In the next step we obtained a total of 223 common miRNAs in H23, 335 in A2780 and 264 in 41 paired cell line. We next selected, as with known miRNAs, those miRNAs overexpressed 4 or more times (Fold Change >2) in resistant exosomes versus sensitive ones: 22 miRNAs in H23, none in A2780 and 4 in 41. In this case, none of these miRNAs were shared by at least two of the cell lines, so we increased the strength of our unknown candidates choosing those with a difference of more than 64 times higher in the resistant compared with the sensitive (Fold Change >6) in at least one of the cell lines: miR-novel-36434, miR-novel-5149 and miR-novel-55745 (names are assigned according with their chromosomal position) (FIG. 5, right panel).

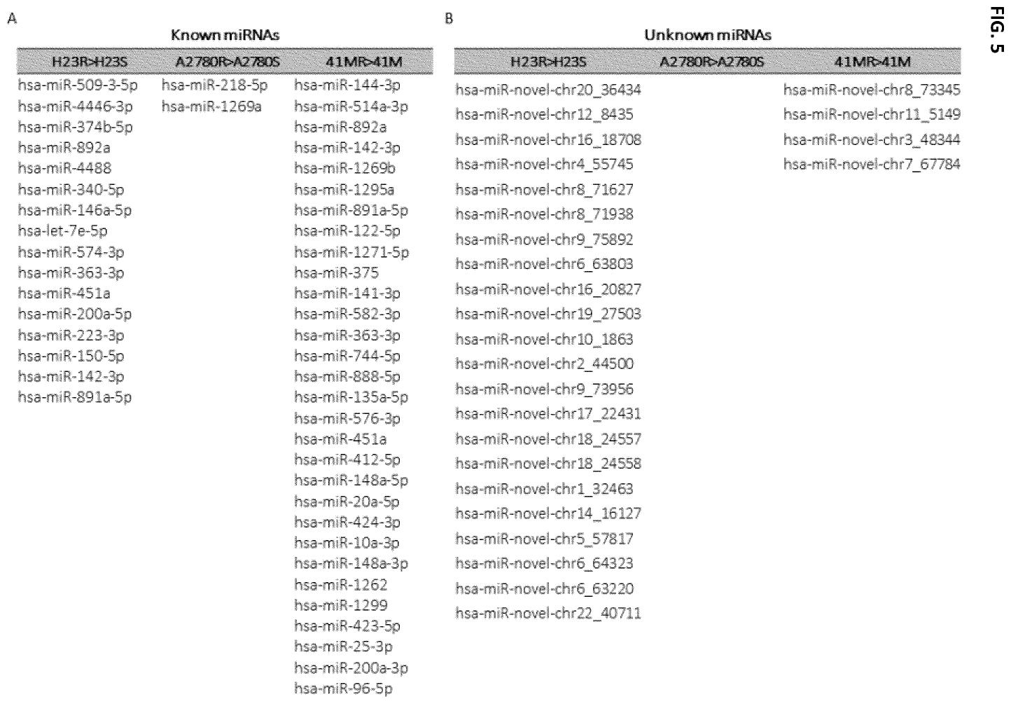

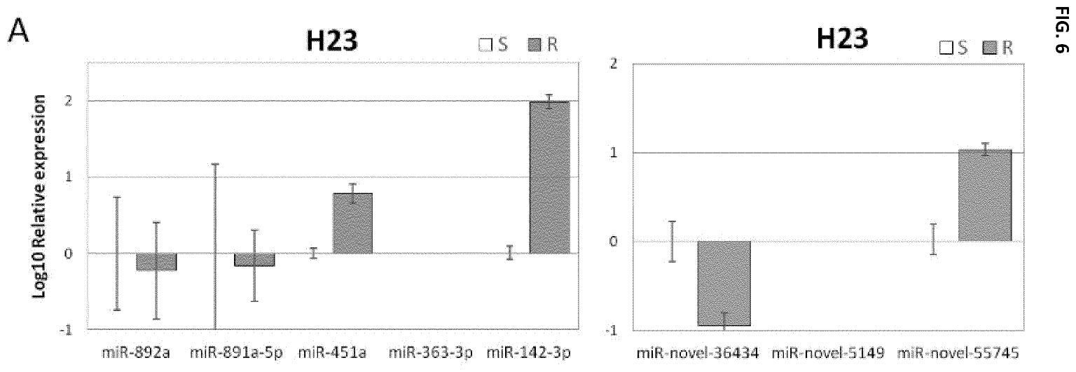

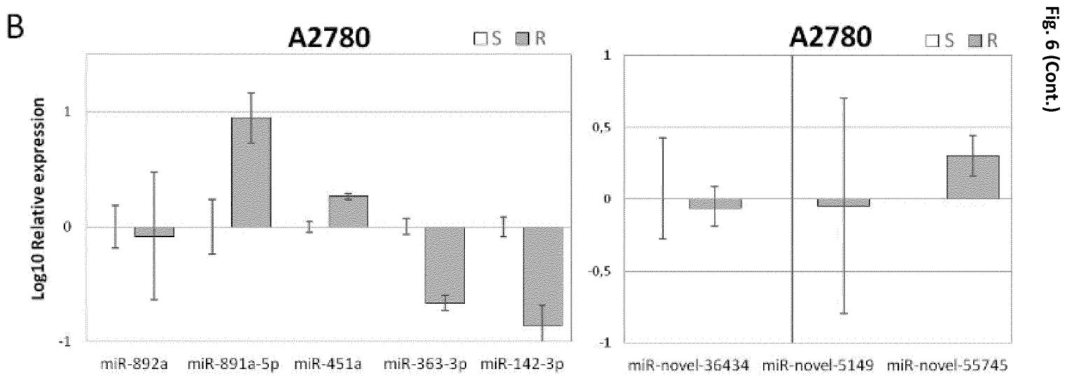

To validate the expression of candidates with an alternative methodology, we performed qRT-PCR using Taqman probes. We evaluated changes in expression or levels of the five known selected miRNAs and the three unknown miRNAs in the three matched cell lines. In relation to known miRNAs, we found a significant expression increase in exosomes from H23R compared with H23S in miR-451a and miR-142-3p, and in miR-891a and miR-451a in the exosomes from the two ovarian cell lines A2780R and 41MR versus their parental subtypes (FIG. 6A-C, left panel). Regarding the unknown miRNAs, miR-55745 was validated in the three cell lines (FIG. 6A-C, right panel). To further determine whether the role of the miRNAs involved in cisplatin resistance miR-451a, 142-3p, 891a and 55745, could be extended to the response to platinum-derived compounds in general, the carboplatin resistant H23R-CBDCA and A2780R-CBDCA cell subtypes, previously established in the laboratory, were used to analyze the expression or levels of the miRNAs validated in CDDP-resistant cell lines by qRT-PCR. The results showed a high expression of miR-142-3p in the H23 line resistant to carboplatin with respect to the sensitive one. However, those differences were not observed for the other two miRNAs 451a and 55745 (FIG. 6D). In the case of carboplatin-resistant line A2780, we observed an increase in the expression of miR-891a and 55745 compared to the sensitive line. These changes were not found for miR-451a (FIG. 6E).

Secondly, to identify exosomal proteins potentially involved in the induction of cisplatin resistance, we analyzed the content of the exosomes from the secretome of paired sensitive and resistant cells H23S/R, A2780S/R and 41M/R with a shotgun proteomic study using liquid chromatography coupled to tandem mass spectrometry (LC-MS/MS). The identification of the proteins from the proteomic results was done using the Mascot search engine and a total of 643 and 664 proteins were obtained in H23S and H23R; 883 and 690 in A2780S and A2780R; and 796 and 705 in 41M and 41MR. In addition, all the samples identified a large number of exosomal markers present in the list of 100 exosomal proteins collected in the ExoCarta database: 62 in H23S, 63 in H23R, 69 in A2780S, 67 in A2780R, 78 in 41M and 66 in 41MR.

For the semiquantitative analysis of the proteins the Scaffold program was used and the 549 proteins identified with at least two peptides were included, to increase the robustness of the analysis. With this approach, the differences between each pair of sensitive and resistant cell lines were estimated based on the number of spectra identified for each peptide. We found 131 over-represented proteins in the exosomes of the resistant subtype H23R, 122 in A2780R and 126 proteins in 41MR, in all cases in comparison with the corresponding sensitive subtype.

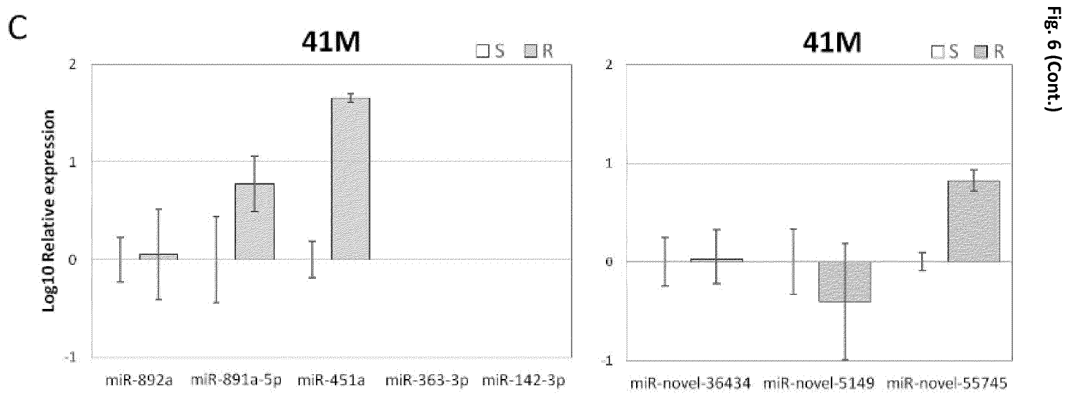

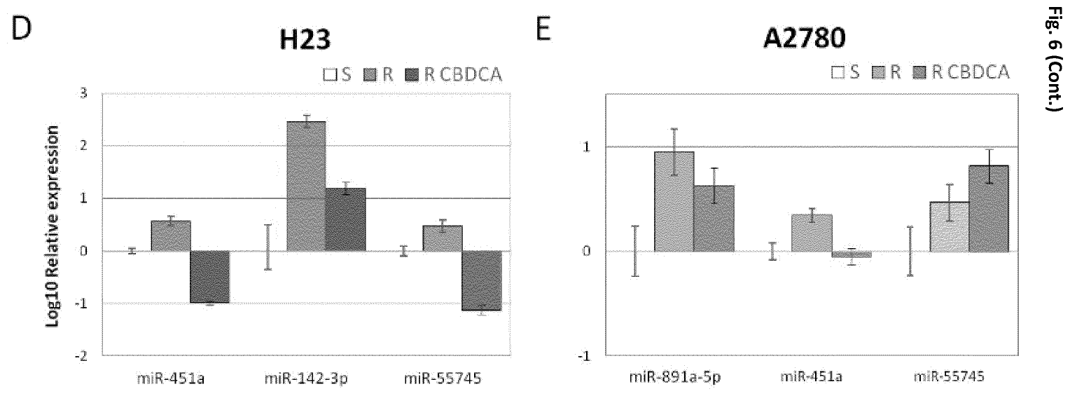

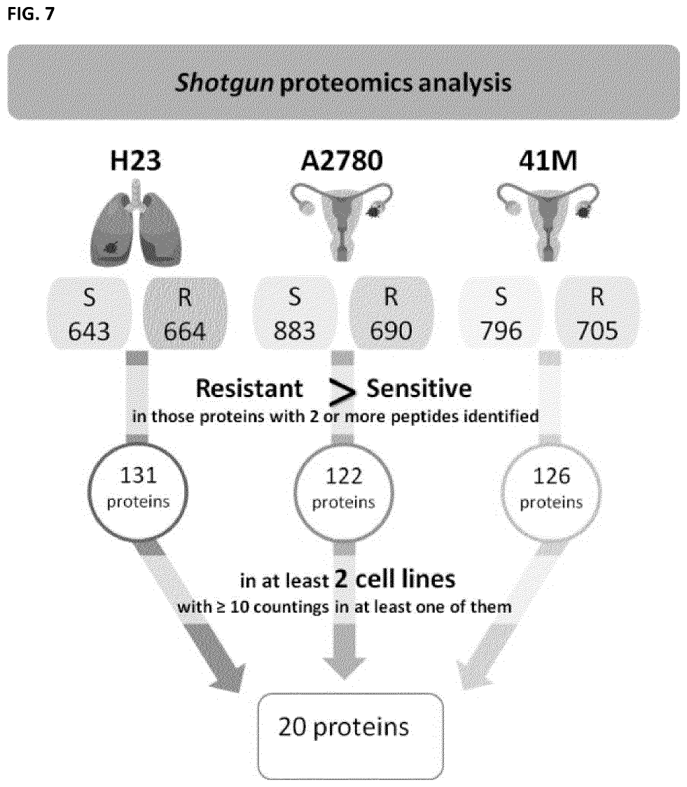

To improve the strength of our selection and find robust candidates for further clinical use, we selected the 20 proteins shared by at least two of the three cell lines, which also had a difference greater than or equal to 10 counts between the two phenotypes in some of the them (FIG. 7).

Translational Validation of Candidates of High Exosomal miR-142 Levels Predict Poor Prognosis in NSCLC in Combination with miR-451a

To determine the clinical relevance of our findings, we collected 51 plasma samples from advanced NSCLC patients (stages III and IV) who had not received any treatment at the time of extraction. After that, all of them received platinum-based chemotherapy as first line of treatment and were clinically followed in order to monitor the time of relapse and overall survival. On the one hand, we extracted total exosomal RNA and analyzed the levels of the miRNAs validated in H23 lung cancer cell line: miR-142, miR-451a and miR-55745 in our NSCLC cohort by qRT-PCR. We divided patients into two groups: high or low levels of the miRNA each case. The cut-off was optimized by performing a COX regression analysis segmenting the cohort in the different quartiles, finally selecting the patients “high-miRNA” and “low-miRNA” according to their position with respect to the 75 percentile in each of the miRNAs. To evaluate the association between miRNAs' levels and prognosis in NSCLC patients we used Kaplan-meier survival analysis. We found that high levels of miR-142 were significantly related with a poor overall survival and progression-free survival (PFS) (FIG. 8A). We also found that the patients with higher levels of exosomal miR-451a showed poorer prognosis in terms of PFS and OS (FIG. 8B). Regarding the unknown miR-55745, no significant differences were observed between low and high groups (FIG. 8C).

However, we found a statistically significant increase (p=0.024) in exosomal miRNA-55745 levels in advanced NSCLC patients compared with levels obtained in a cohort of 10 healthy controls (FIG. 9). Furthermore, to investigate the strength of the combination of both miRNAs 142 and 451a in the prognosis prediction, we analyzed PFS and OS dividing patients in three groups: high levels of both miRNAs, low levels of both miRNAs and the rest with only one of them high or low. Our findings showed that the risk of relapse increases more than 9 times and the risk of exitus more than 10 times in patients with high levels of both miRNAs (FIG. 10).

On the other hand, we extracted exosomal proteins from 45 samples of our cohort of advanced NSCLC patients by size exclusion chromatography. We performed a Targeted Mass Spectrometry Analysis of the 20 selected proteins in the cell lines and we found presence of 10 of them inside the circulating exosomes from the patients. In the same way as with miRNAs, survival analyzes using Kaplan-Meier curves were done by comparing two groups: patients with “high levels” or “low levels” of each of the proteins. In this case, the cut was placed on the average of the levels of each protein in the cohort. We observed the existence of a statistically significant relationship between elevated exosomal levels of the ITIH1 protein and a worse prognosis in terms of PFS (p=0.020) and OS (p=0.012) (FIG. 11A). In the same way, high levels of FBN2 influenced a worse PFS (p=0.013) and OS (p=0.001) (FIG. 11B). In addition, we observed that the LOXL2 and gelsolin proteins had the same distribution pattern in the patients and that those with high levels of these two proteins had lower progression-free survival (p=0.002) and overall survival (p=0.001) (FIG. 11C). No significant differences were observed in terms of PFS or OS with the rest of the proteins: TSP1, APOE, TNC, HBA, ITIH2 and A2M.

1. An in vitro use of the levels of miR-142, miR-451a, or any combination thereof, isolated or present inside circulating exosomes of a sample isolated from serum, blood or plasma of a human subject diagnosed with Non-small cell lung carcinoma, for the purpose of predicting progression-free survival in that subject or to predict the overall survival of the subject.

2. An in vitro use of the levels of miR-142 and miR-451a, isolated or present inside circulating exosomes of a sample isolated from serum, blood or plasma of a human subject diagnosed with Non-small cell lung carcinoma, for the purpose of predicting an increased risk of relapse or an increased risk of exitus.

3. The in vitro use according to any of claims 1 to 2, wherein the subject suffering from Non-small cell lung carcinoma is treated with an alkylating agent, wherein preferably said alkylating agent is selected from the list consisting of: nitrogen mustards such as mechlorethamine, cyclophosphamide, ifosfamide, melphalan, or chlorambucil; ethyleneimines and methylmelamines such as altretamine; methylhydrazine derivatives such as procarbazine; alkyl sulfonates such as busulfan; nitrosoureas such as carmustine; triazenes such as dacarbazine or temozolomide; or platinum coordination complexes such as cisplatin, carboplatin, oxaliplatin, dicycloplatin, eptaplatin, lobaplatin, Miriplatin, Nedaplatin, Oxaliplatin, Picoplatin, Satraplatin, Triplatin or tetra nitrate.

4. The in vitro use according to any of claim 1 or 3, wherein the upregulation of the levels of miR-142, miR-451a, or any combination thereof, isolated or present inside the circulating exosomes is indicative of a poor overall survival or of a poor PFS.

5. The in vitro use according to claim 2, wherein the up regulation of the levels of miR-142 and miR-451a, isolated or present inside the circulating exosomes is indicative of an increased risk of relapse or an increased risk of exitus.

6. The in vitro use according to any of claims 1 to 5, wherein the levels of miR-142 and miR-451a are normalized with respect to the exosomal content of miR-151a.

7. The in vitro use according to any of claims 1 to 6, wherein the prediction is determined by comparing the levels of miR-142, miR-451a, or any combination thereof, with a threshold or cutoff level, wherein preferably such threshold or cutoff level is obtained from a group of healthy controls or corresponds to, preferably the 75th percentile value of, the normalized levels of the amount of exosomal miR-142-3p and miR-451a (21.35 2^{-ΔCt} and 1258.07 2^{-ΔCt} respectively) versus miR-151a in NSCLC patients

8. A method to monitor the progression of the oncological disease of a subject diagnosed with NSCLC, wherein such a method comprises the following steps:
a. determining the levels of miR-142, miR-451a, or any combination thereof, isolated or present inside circulating exosomes of a sample isolated from serum, blood or plasma of a human subject diagnosed with Non-small cell lung carcinoma; and
b. Determining the subject's disease monitoring or progression based on determination carried out in step (a),
wherein the up regulation of the levels of miR-142 or miR-451a or any combination thereof isolated or present inside the circulating exosomes is indicative of a poor overall survival or of a poor PFS of the subject, and wherein the upregulation of the levels of miR-142 and miR-451a isolated or present inside the circulating exosomes is indicative of an increased risk of relapse and an increased risk of exitus, and wherein such determination is made by comparing the levels of miR-142, miR-451a, or any combination thereof, with a threshold or cutoff level, wherein preferably such threshold or cutoff level is obtained from a group of healthy controls or corresponds to, preferably the 75th percentile value of, the normalized levels of the amount of exosomal miR-142-3p and miR-451a (21.35 2^{-ΔCt} and 1258.07 2^{-ΔCt} respectively) versus miR-151a in NSCLC patients.

9. A method for predicting the response to treatment of a subject treated with an alkylating agent, preferably selected from the list consisting of: nitrogen mustards such as mechlorethamine, cyclophosphamide, ifosfamide, melphalan, or chlorambucil; ethyleneimines and methylmelamines such as altretamine; methylhydrazine derivatives such as procarbazine; alkyl sulfonates such as busulfan; nitrosoureas such as carmustine; triazenes such as dacarbazine or temozolomide; or platinum coordination complexes such as cisplatin, carboplatin, oxaliplatin, dicycloplatin, eptaplatin, lobaplatin, Miriplatin, Nedaplatin, Oxaliplatin, Picoplatin, Satraplatin, Triplatin or tetranitrate, wherein such subject is diagnosed with NSCLC, and wherein such method comprises the following steps:
a. Performing the methodology according to claim 8; and
b. Determining the response to treatment of the subject's disease based on the upregulation of any of miR-142 or miR-451a,
wherein a higher level of any of miR-142 or miR-451a or any combination thereof, in comparison to a sample obtained from the subject previously or with respect to a reference value or with a threshold or cutoff level, wherein preferably such threshold or cutoff level is obtained from a group of healthy controls or corresponds to the 75th percentile value of the normalized levels of the amount of exosomal miR-142-3p and miR-451a (21.35 2^{-ΔCt} and 1258.07 2^{-ΔCt} respectively) versus miR-151a in NSCLC patients, is indicative of an unfavourable response to treatment.

10. A method for predicting the response before the onset of treatment of a subject with an alkylating agent, preferably selected from the list consisting of: nitrogen mustards such as mechlorethamine, cyclophosphamide, ifosfamide, melphalan, or chlorambucil; ethyleneimines and methylmelamines such as altretamine; methylhydrazine derivatives such as procarbazine; alkyl sulfonates such as busulfan; nitrosoureas such as carmustine; triazenes such as dacarbazine or temozolomide; or platinum coordination complexes such as cisplatin, carboplatin, oxaliplatin, dicycloplatin, eptaplatin, lobaplatin, Miriplatin, Nedaplatin, Oxaliplatin, Picoplatin, Satraplatin, Triplatin or tetranitrate, wherein such subject is diagnosed with NSCLC, and wherein such method comprises the following steps:
a. Performing the methodology according to claim 8; and
b. Determining the predictive response to treatment of the subject's disease based on the upregulation of any of miR-142 or miR-451a,
wherein a higher level of any of miR-142 or miR-451a or any combination thereof, in comparison to a sample obtained from the subject previously or with respect to a reference value, is indicative of an unfavourable response to treatment.

11. Use of a kit comprising reagents and means for detecting any of miR-142 or miR-451a, to implement the methodology according to any of claims 8 to 10.
